(19) United States
(12) Patent Application Publication
(10) Pub. No.: US 2022/0339178 A1
(43) Pub. Date: Oct. 27, 2022

(54) COMPOSITIONS AND METHODS FOR COGNITIVE PROTECTION OF POLLINATORS AGAINST PESTICIDES

(71) Applicants: Arizona Board of Regents on Behalf of the University of Arizona, Tucson, AZ (US); Colegio Mayor de Nuestra Senora del Rosario, Bogota (CO); Pontificia Universidad Javeriana, Bogota (CO)

(72) Inventors: Andre Josafat Riveros Rivera, Bogota (CO); Wulfila Gronenberg, Tucson, AZ (US); Carlos Fabian Morantes-Ariza, Bogota (CO); Lina Maria Garcia Forero, Bogota (CO)

(21) Appl. No.: 17/640,756

(22) PCT Filed: Sep. 4, 2020
(86) PCT No.: PCT/US2020/049457

§ 371 Date: Mar. 4, 2022

Related U.S. Application Data
(60) Provisional application No. 62/896,944, filed on Sep. 6, 2019.

(51) Int. Cl. A61K 31/7048; A61K 31/192; A61K 31/352; A61P 39/02; A61P 25/00

(52) U.S. Cl. CPC A61K 31/7048; A61K 31/192; A61K 31/352; A61P 39/02; A61P 25/00

(57) ABSTRACT

Compositions and methods for protecting insect pollinators against the harmful cognitive effects of pesticides are disclosed. The compositions are suitable for administration to or ingestion by an insect pollinator and contain one or more phenolic compounds, such as the flavonoids quercetin, rutin, kaempferol and p-coumaric acid in an effective amount to protect against pesticide-induced impairment of a cognitive function of the insect pollinator. Methods of using such compositions for preventing or reducing pesticide-induced impairment of cognitive functions including learning, memory, decision-making, motor activity, and navigation in an insect pollinator are also provided. The compositions and methods protect insect pollinators including honeybees and bumblebees against neurotoxic pesticides with different pharmacodynamics including neonicotinoids, such as Imidacloprid, fipronil, and pyrethroids, such as deltamethrin.

### CROSS-REFERENCE TO RELATED APPLICATION

This application claims benefit of and priority U.S. Provisional Application No. 62/896,944, filed Sep. 6, 2019, and which is specifically incorporated by reference herein in its entirety.

### FIELD OF THE INVENTION

The invention is generally related to insect pollinators (e.g., honeybees), and more particularly to compositions and methods for prophylactically protecting insect pollinators against the negative effects of neurotoxic pesticides.

### BACKGROUND OF THE INVENTION

Insects pollinate greater than 70% of food crops, contributing an estimated U.S. $215 billion to the global economy each year. Besides contributing to crop yield, insect pollinators can also improve the quality of the harvest. Insect pollination also provides ecosystem services that underpin biodiversity. Due to their clear importance in food security, global economics, and ecosystem stability, there is worldwide concern over the decline in insect pollinators, including wild and managed bees.

Known risks to insect pollinators include parasites, diseases, habitat loss, poor nutrition, and exposure to pesticides. Pesticide exposure is a major contributor to the current decline in populations of pollinating insects, which provide essential pollination services for food production. In the past several decades, there has been a rapid increase in the use of pesticides that target the insect nervous system (neuropesticides) and are the principal means to control insect pests of crops, livestock, pets, and people. While neuropesticides, such as neonicotinoids, may be used as systemic insecticides with improved selectivity for insects relative to vertebrates, non-target pollinators may be adversely affected via direct contact or by consumption of contaminated nectar and pollen.

Neonicotinoids target the nicotinic receptors for acetylcholine (nAChR) (Matsuda K., et al., Mol. Pharmacol., 76(1):1-10. 2009) broadly present in the central nervous system (CNS) of insects (Dupuis J., et al., Neurosci. Biobehav. Rev., 36(6):1553-64. 2012). Within the CNS, the mushroom bodies (MBs) (areas involved in learning, memory, navigation, sleep and walking) are rich in nAChR (Reviewed in Schuermann F W., Arthropod Struct. Dev., (5):399-421 2016), and therefore are targeted by neonicotinoids. It is widely accepted that very low levels of neonicotinoids exist in nectar (1.9 ppb) and pollen (6.1 ppb) (Moffat C., et al., FASEB J., 29(5):2112-9. 2015). Whereas full toxicity leads to death, sublethal doses profoundly impair behavior. Sublethal effects are of major concern as non-targeted visitors, such as pollinators, are exposed to low quantities of neonicotinoids from nectar, pollen and dust drift, which are sufficient to affect their physiology and behavior.

In fact, evidence indicates that sublethal levels of neonicotinoids may cause deficits in brain function, olfactory learning, navigation, and colony development (Moffat C., et al., FASEB J., 2015), therefore implicating their use in bee decline. For example, exposure to Imidacloprid, a first-generation neonicotinoid used worldwide, impairs learning and memory (Decourtye A., et al., Ecotoxicol. Environ. Saf., 57(3):410-9. 2004; Williamson S M., et al., Invert. Neurosci., 13(1):63-70. 2013), motor activity (Lambin M., et al., Arch. Insect. Biochem. Physiol., 48(3):129-34. 2001; Williamson S M., et al., Ecotoxicology, 23(8):1409-18. 2014), sucrose sensitivity (Aliouane Y., et al., Environ. Toxicol. Chem., 28(1):113-22. 2009) and navigation abilities (Fischer J., et al., PloS one, 9(3):e91364. 2014). These cognitive impairments may derive from structural degeneration of the so-called microglomeruli (Peng Y C & Yang E C, Sci. Rep., 6:19298. 2016) likely due to increases in oxidative stress (Malev et al., Pesticide biochemistry and physiology, 104(3):178-186. 2012).

Following the evidence of these negative effects, certain neonicotinoids were banned in Europe (Butler D., Nature, 555(7695):150-151. 2018). Nevertheless, neonicotinoids play a major role in control of pests and concerns have been raised against a generalized ban from market since farmers may return to the older pesticides, which were even more toxic to bees. Concerns have also been raised that in the wake of such bans, there could be development of resistance by pests that are treated with other pesticides, such as pyrethorids. Thus, policy makers need to finely balance food security from the perspectives of pollinator disappearance and pest outbreaks.

Accordingly, there remains a need for compositions and methods to circumvent the negative impact of pesticides on insect pollinators while controlling pests.

It is therefore an object of the invention to provide compositions and methods for protecting insect pollinators from pesticides.

It is another object of the invention to provide compositions and methods for enhancing the cognitive function of insect pollinators.

### SUMMARY OF THE INVENTION

Compositions and methods for protecting insect pollinators against the harmful neurocognitive effects of pesticides are disclosed. Provided herein are compositions suitable for administration to or ingestion by an insect pollinator including one or more phenolic compounds in an effective amount to protect against impairment of a cognitive function of the insect pollinator. The compositions may also contain a source of carbohydrates, proteins, lipids, vitamins, minerals, water or combinations thereof. Exemplary sources include natural or artificial nectar, honey, sugar, sugar syrup, pollen or pollen substitute, soy flour, soy meal, gluten (e.g., soy or corn), skim milk, yeast, pollard, oil, and combinations thereof. In some embodiments, the sources can be or can be derived from commercially available bee feed such as AP23®, BEE-PRO®, FEEDBEE, MEGABEE and ULTRA BEE.

In preferred embodiments, the one or more phenolic compounds are flavonoids, for example, flavones, flavanones, flavonols, isoflavones, anthocyanins, flavanols (catechins), chalcones, or neoflavonoids. Preferred flavonols include quercetin, rutin, myricetin, kaempferol, fisetin, morin, isorhamnetin, and derivatives or variants thereof.

In some embodiments, the one or more phenolic compounds is isoquercetin, rutin, quercetin, quercitrin, hyperoside, quercimeritrin, baimaside, querciturone, 3-O-methylquercetin, quercetin 3-sambubioside, miquelianin, spiraeoside, isorhamnetin, quercetin 3′-O-sulfate, quercetin 7-O-glucoside, quercetin 3-O-Rhamnoside, quercetin 3,4′-diglucoside, quercetin 3-sophorotrioside, or a combination thereof.

The disclosed compositions can be used as a food, feed additive or supplement, or nutraceutical. In some embodiments, the insect pollinators to which the compositions are provided include, but are not limited to, butterflies, moths, flies, beetles, wasps and bees (e.g., honeybee, bumblebee, carpenter bee, leafcutter bee, blueberry bee, squash bee, mason bee, orchid bee, stingless bee, and sweat bee). The preferred embodiments, the insect pollinators to which the compositions are administered include or consist of bees, particularly honeybees and/or bumblebees. The compositions can be administered or provided to the insect pollinator via ingestion.

The disclosed compositions can be used to protect against impairment of various cognitive functions, including but not limited to, those involved in information processing such as learning, memory, navigation, motor activity, sucrose sensitivity, and combinations thereof. The amount of the composition administered can be effective to prevent or reduce loss of or reduction in memory, learning, navigation skills, motor activity, or combinations thereof in the insect pollinator, for example, upon exposure to a pesticide. The amount of the composition administered can be effective to prevent or reduce loss of or reduction in memory, learning, navigation skills, motor activity, or combinations thereof in the insect pollinator as compared to an insect pollinator not administered the composition. The amount of the composition administered can be effective to prevent or reduce mitochondrial dysfunction, apoptosis and/or oxidative stress in the brain, for example the mushroom bodies and the antennal lobes.

Impairment of cognitive function can be induced by exposure to a pesticide. Preferably, the pesticide is at a sublethal dose (e.g., field dose). The pesticide can be neurotoxic. For example, the pesticide may adversely affect cholinergic, GABAergic or glutamatergic neurotransmission, brain areas such as the mushroom bodies, or lower levels such as mitochondrial function; increase apoptosis; increase oxidative stress; or combinations thereof. Preferably, the pesticide targets (e.g., is an agonist, partial agonist, antagonist or inhibitor of) the nicotinic acetylcholine receptor (nAChR), GABAergic receptors, glutamatergic receptors or voltage-gated sodium channels (Na⁺ᵥ). Exemplary pesticides include neonicotinoids (for example imidacloprid, thiacloprid, clothianidin, thiamethoxam, acetamiprid, nitenpyram, dinotefuran, and nithiazine), fipronil, sulfoximines derivatives (for example sulfoxaflor), pyrethroids (for example deltamethrin) and glyphosate. In some embodiments, the pesticide can be a carbamate or organophospate. In preferred embodiments, the pollinators are exposed to the pesticide directly (for example spraying) or indirectly (for example in nectar or pollen after exposure to drift) after administration of the phenolic compound(s) or composition.

The disclosed compositions, including those mentioned above and in more detail below, can be employed in various methods of use. For example, methods of protecting the cognitive function of an insect pollinator by administering a phenolic compound, or a composition including a phenolic compound, to an insect pollinator in need thereof are provided. Methods of protecting the learning and/or memory capabilities of an insect pollinator by administering a phenolic compound, or a composition including a phenolic compound, to the insect pollinator are provided. Methods for preventing or reducing impairment of cognitive function in an insect pollinator by administering to the insect a phenolic compound, or a composition including a phenolic compound, are disclosed.

Methods of controlling insect pests are also disclosed. The methods involve treating an area occupied by insect pests and insect pollinators with a pesticide, and administering to the insect pollinator a composition including an effective amount of one or more phenolic compounds to prevent or reduce impairment of a cognitive function and/or increase a cognitive function in the insect pollinator. The area can be treated with the pesticide before the composition is administered to the insect pollinators. Alternatively, the area can be treated with the pesticide after the composition is administered to the insect pollinators. In some embodiments, the area is treated with the pesticide by foliar application, soil injection, tree injection, ground application as a granular or liquid formulation, or as a pesticide-coated seed treatment. Preferably, the area is treated with an effective amount of pesticide to reduce the number of insect pests in the area. The pesticide can be lethal to insect pests and/or in an amount that is sublethal to insect pollinators in the area or near the area of administration (e.g., within an area effected by dust drift of the pesticide). Exemplary insect pests that can be targeted include, but are not limited to, one or more of aphids, thrips, whiteflies, mites, leafhoppers, mealybugs, spittlebugs, fleas, termites, scales (e.g., armored scales, soft scales), and beetles.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIGS. 1A-1C show evaluation of learning and memory in the Africanized honey bee. FIG. 1A is a bar graph showing a measure of learning (score 0-7); FIG. 1B is a bar graph showing a measure of memory (score 0-100); and FIG. 1C is a bar graph showing a measure of latency of response in honey bees fed with sugar-water, Quercetin, Rutin, Imidacloprid, Rutin and Imidacloprid, or Quercetin and Imidacloprid (see Example 1).

FIGS. 2A-2B are bar graphs showing a measure of learning (score 0-10) (FIG. 2A) and memory (score 0-100) (FIG. 2B) in bumble bees fed with sugar water, Rutin, Imidacloprid, or Rutin and Imidacloprid (see Example 2). FIG. 2C is a line graph showing % proboscis extension response (PER) during acquisition for bumble bees fed with sugar water, Rutin, Imidacloprid, or Rutin and Imidacloprid. FIG. 2D is a bar graph showing log latency for bumblebees fed with sugar water, Rutin, Imidacloprid, or Rutin and Imidacloprid (see Example 2).

FIG. 3 is a bar graph showing the % flies above the motor activity threshold for Control, Imid, Rut 0.1 μl, Rut 0.1 μl+Imid, Rut 1 μl, Rut 1 μl+Imid, Rut 10 μl, Rut 10 μl+Imid (see Example 3).

FIGS. 4A-4B are bar graphs showing the evaluation of learning (FIG. 4A) and memory (FIG. 4B) in bumblebees feeding ad libitum Rutin or sucrose water and then administered imidacloprid or fipronil (see Example 4). Numbers in each bar are sample size per treatment for learning and memory.

FIG. 5A is a bar graph showing the total PER score as a measure of responsiveness to sucrose water during the evaluation of the protective effect of two flavonoids against the impairment produced by a pyrethroid. FIGS. 5B-5E are line graphs showing the % of response to different concentrations of sucrose water across treatments and across time (see Example 5).

FIGS. 6A-6B are bar graphs showing the evaluation of learning (FIG. 6A) and memory (FIG. 6B) in honey bees for the protective effect of flavonoids and a phenolic acid against impairment by Fipronil. Significant differences are indicated relative to Control bees (see Example 6).

FIGS. 7A-7B are bar graphs showing the evaluation of learning (FIG. 7A) and memory (FIG. 7B) in bumble bees for the protective effect of self-administration (bees were able to drink from a feeder) of a flavonoid against impairment by chronic administration of fipronil. Numbers in each bar are sample size per treatment for learning and memory (see Example 7).

FIGS. 8A-8D are curves showing the distribution of the conformational states of nAChR when coupled with Imidacloprid, Acetylcholine, Quercetin, Rutin, and 4′-Phenylflavone.

FIG. 9 is a line graph showing the radius of the nAChR pore. The dots mark the position for the most frequent occurrence of a conformational state of the channel when it is coupled to each ligand. Imidacloprid (IMI), Acetylcholine (ACh), Quercetin (Quer), Rutin (Rut), and 4′-Phenylflavone (4-Phenyl).

FIG. 10 is a model comparing the recostructed nAChR from A. mellifera in a closed and an open state. Bottom-left indicates the intracelular domain of the protein.

### DETAILED DESCRIPTION OF THE INVENTION

### I. Definitions

As used herein, the term “bee” refers to members of the Family Apidae, Order Hymenoptera. A “honey bee” refers to members of the genus, Apis. For example, the domesticated honeybee is Apis mellifera. A “bumble bee” (also referred to herein and elsewhere as “bumblebee”) refers to the genus ‘Bombus’. For example, domesticated species of bumble bee include the buff-tailed bumble bee, Bombus terrestris and the bumble bee Bombus impatiens.

As used herein, the term “pollinator” means an animal that moves pollen from the male anther of a flower to the female stigma of a flower, which helps bring about fertilization of the ovules of the flower by the male gametes in the pollen grains. “Insect pollinators” are insects whose behavior results in pollination of one of more species of plant. The term does not denote a particular age or sex.

As used herein, the terms “pest,” and “insect pest” mean an insect or arthropod that damages agricultural products or reduces agricultural yield of agricultural products that are economically useful or that find desirable utility in human or animal consumption. However, “pest” is also understood as any arthropod or insect that is destructive by infesting and damaging pollinating insects, bee hives, or reducing honey bee populations, or by causing a reduction in honey production.

As used herein, the term “exposure” when used in the context of a pesticide describes the state of having contact with the pesticide. Exposure can be obtained through a variety of mechanisms. For example, an insect pollinator can be exposed to a pesticide through food or water ingestion, nesting material (e.g., resin, wax etc.), contact with spray drift and dust drift generated by pesticide application, contact with contaminated plants, soil, water, and inhalation.

As used herein, the term “sublethal” describes the amount or concentration of pesticide that does not give rise to acute mortality. Sublethal effects on an insect may involve modifications of insect (e.g., honeybee) behavior and physiology (e.g., immune system). They do not directly cause the death of the individual or the collapse of a colony but may become lethal in time and/or may make the colony more sensitive (e.g., more prone to diseases), which may contribute to its collapse. For instance, an individual with memory, orientation or physiological impairments might fail to return to its hive, dying from hunger or cold. In the context of neonicotinoids, a sublethal dose can be a field dose, which is an amount or concentration of the pesticide that an insect pollinator may be exposed to during normal foraging. In some embodiments, a field dose of imidacloprid is about 1 μg/L.

As used herein, the term “cognitive function” when used in the context of an insect pollinator, encompasses acquisition, storage, processing, retrieval and use of information, and hence include perception, attention, memory, learning, motor skills, and navigation.

As used herein, the term “impair” in the context of a biological function or parameter means to weaken, reduce or otherwise adversely alter that function or parameter.

As used herein, “mitochondrial dysfunction” describes a state in which any of the typical mitochondrial processes or functions are eliminated, hindered, or reduced. For example, the term encompasses a mitochondrial state characterized by reduced electron transport, reduced ATP production, or altered mitochondrial membrane potential. The term can refer to any loss of function in the mitochondria. At the molecular level, a reduction in mitochondrial function typically occurs as a result of (1) a loss of maintenance of the electrical and chemical transmembrane potential of the inner mitochondrial membrane, (2) alterations in the function of the electron transport chain, or (3) a reduction in the transport of critical metabolites into mitochondria.

As used herein, the term “neurotoxic” means poisonous or otherwise harmful to the nervous system and the functions associated to it, for example neuromotor control.

As used herein, “effective amount” means that the amount of the composition used is of sufficient quantity/concentration to affect an intended response. For example, the amount of the disclosed neuroprotective compositions can be effective to ameliorate one or more symptoms or effects of a pesticide on an insect pollinator. Non-limiting examples of symptoms or effects of pesticides include reduction in information processing including, for example, learning, memory, motor control, decision-making and/or navigation. Such amelioration only requires a reduction or alteration, not necessarily elimination. The precise dosage will vary according to a variety of factors such as species, subject-dependent variables (e.g., organism size, age, immune system health, etc.), the pesticide being protected against, as well as the route of administration and the pharmacokinetics and pharmacodynamics of the agent being administered. Alternatively, the amount of pesticide can be effective to reduce the number of insect pests in a treated area. In some examples, the amount of pesticide is effective to kill (e.g., be lethal to) insect pests.

As used herein, the term “reduce” means to decrease an activity, function, response, condition, disease, or other biological parameter. This can include, but is not limited to, the complete ablation of the activity, function, response, condition, or disease. This may also include, for example, at least a 5% decrease in the activity, function, response, condition, or disease, or other biological parameter as compared to a native or control level (e.g., before or after administration of a disclosed composition or levels in an insect pollinator not administered a disclosed composition). Thus, the reduction can be a 5, 10, 20, 30, 40, 50, 60, 70, 80, 90, 100%, or any amount of decrease in between as compared to native or control levels.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D.

Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Compositions

In recent years, there has been an increased concern regarding the sublethal effects of neuropesticides on insect pollinators and the consequences on agricultural biodiversity and sustainable agriculture, and ultimately, food security. Neonicotinoids, for example, impact the neural and immune systems, leading to cognitive impairment and higher vulnerability to parasites. Affected cognitive processes include learning (Stanley D., et al., Sci Rep., 5:16508. 2015), memory (Wright G., et al., Sci Rep., 5:15322. 2015) and navigation (Fischer J., et al., PloS one, 9(3):e91364. 2014; Henry M., et al., Science, 336(6079):348-50. 2012) presumably due to effects on areas such as the mushroom bodies (Palmer M J., et al., Nat. Commun., 4:1634. 2013), major areas of information processing in the insect brain. Mushroom bodies are rich in nAChr and their function and structure are impaired by neonicotinoids (Moffat C., et al., Sci. Rep., 6:24764. 2016; Buckingham S D., et al., J. Exp. Biol., 200:2685-2692. 1997).

When neonicotinoids were first used, beekeepers started describing different disorders and signs ranging from: bees not returning to the hive, disoriented bees, bees gathered close together in small groups on the ground, abnormal foraging behavior, the occurrence of massive bee losses in spring, queen losses, increased sensitivity to diseases and colony disappearance (Van der Sluijs, J P., et al., Current Opinion in Environmental Sustainability, 5(3-4), 293-305. 2013). Thus far, most efforts have focused on exposing the negative effects of neonicotinoids aiming to ban usage of neonicotinoid pesticides and/or promote transition to pollinator-friendly alternatives to neonicotinoids.

Alternatively, the disclosed compositions and methods can be used to minimize the harmful effect of neonicotinoids and other neuropesticides on insect pollinators such as honeybees and bumblebees. It has been discovered that insect pollinators can be prophylactically protected against the sublethal effects on learning and memory of the broadly used neonicotinoid, imidacloprid. The data in the Examples show, using the Africanized honey bee Apis melhfera as a model, that the flavonoids rutin and quercetin confer significant protection for learning, while rutin protected memory levels. It was observed that, relative to control, both flavonoids enhance learning but not memory whereas imidacloprid lowers learning performance and memory. Bees fed with flavonoids, which subsequently received imidacloprid, exhibited learning performances significantly higher than bees receiving only imidacloprid. Similarly, as a second example, bumblebees Bombus impatiens prophylactically fed with Rutin were protected against imidacloprid in both learning and memory. These results set a foundation for development of pharmacological and/or nutritional alternatives for protection of pollinators from neurotoxic pesticides.

Accordingly, compositions and methods for protecting insect pollinators against the harmful effects of pesticides are disclosed. The disclosed compositions typically include one or more phenolic compounds in an effective amount to protect against impairment of a cognitive function of the insect pollinator. The compositions can contain a source of carbohydrates, proteins, lipids, vitamins, minerals, water or combinations thereof, such as, for example, natural or artificial nectar, honey, sugar, sugar syrup, pollen or pollen substitute, soy flour, soy meal, gluten (e.g., soy or corn), skim milk, yeast, pollard, oil, and combinations thereof. In some embodiments, the source of carbohydrates, proteins, lipids, vitamins, minerals, or water can be, or can be derived from, a commercially available feed such as AP23©, BEE-PRO®, FEEDBEE, MEGABEE and ULTRA BEE.

A. Phenolic Compounds

The disclosed compositions typically include one or more phenolic compounds in an effective amount to protect against impairment of a cognitive function of the insect pollinator. For examples, compositions containing one or more phenolic compounds in an effective amount to prevent reduction in memory, learning, navigation skills, motor activity, or combinations thereof in the insect pollinator upon exposure to a pesticide are provided. Also provided are compositions containing one or more phenolic compounds in an effective amount to reduce or diminish loss of memory, learning, navigation skills, motor activity, or combinations thereof in the insect pollinator upon exposure to a pesticide. Also provided are compositions containing one or more phenolic compounds in an effective amount to enhance cognitive function (e.g., learning) in an insect pollinator.

As used herein, phenolic compound refers to a compound possessing an aromatic ring bearing one or more hydroxyl groups, including their functional derivatives (Jianmei Y., et al., In: Polyphenols Agricultural By-Products as Important Food Sources of Polyphenols. 2014). The term specifically encompasses polyphenols which are compounds that have more than one phenolic hydroxyl group attached to one or more benzene rings. Polyphenols are secondary metabolites of plants. They are produced by the plant defense system to protect plants from invading insects and microorganisms, and to give the plants their specific organoleptic properties such as color, taste and flavor. Though not officially classified as human nutrients, polyphenols play important roles in human health and, therefore, are called nutraceuticals.

Types and contents of phenolic compounds in different foods vary greatly, depending on the type of food, environmental conditions of product growth, and processing/cooking conditions. Rich sources of dietary polyphenols include various spices and dried herbs, cocoa products, some darkly colored berries, some seeds (e.g., flaxseed) and nuts (e.g., chestnut, hazelnut) and some vegetables, including olive and globe artichoke heads. Medicinal herbs and spices also contain different types of health promoting phenolics and some agricultural by-products/residues such as apple pomace, cranberry pomace, grape pomace, citrus peels, peanut skin, soy pulp/okara and sweet potato peels usually contain high levels of polyphenols. For review, see Jianmei Y., et al., In: Polyphenols Agricultural By-Products as Important Food Sources of Polyphenols. 2014.

In food science, natural polyphenols are generally classified into groups and sub-classes based on the similarity of their chemical structures. Four major classes of polyphenols found in foods are phenolic acids, flavonoids, lignans, and stilbenes. Accordingly, in some embodiments, the disclosed compositions include one or more phenolic compounds such as, phenolic acids, flavonoids, lignans, and stilbenes. In a particular embodiment, the disclosed compositions include one or more phenolic compounds such as, phenolic acids, flavonoids, lignans, and stilbenes in an effective amount to protect against impairment of a cognitive function of the insect pollinator.

### Flavonoids

In preferred embodiments, the one or more phenolic compounds are flavonoids. All flavonoids possess a three-ring diphenylpropane (C₆C₃C₆) core structure (e.g., Formula I or II).

The basic structure includes the fused A and C ring, with the phenyl ring B attached—through its 1 position to the 2-position of the C ring (numbered from the pyran oxygen).

The general structure of flavonoids is depicted as Formulas I and II.

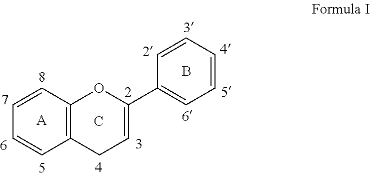

(as described in Jianmei Y., et al., In: Polyphenols Agricultural By-Products as Important Food Sources of Polyphenols. 2014, which is specifically incorporated by reference herein in its entirety).

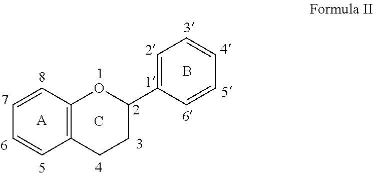

(as described in Kumar and Pandey, Chemistry and Biological Activities of Flavonoids: An Overview, Volume 2013 IArticle ID 162750 I, 16 pages, doi.org/10.1155/2013/162750, which is specifically incorporated by reference herein in its entirety).

Typical modifications of the basic core structure include hydroxylation and/or methylation at positions C-3, C-5, C-7, C-3′, C-4′, and/or C-5′. Other modifications include acylations, sulfonations and prenylations. Flavonoids occur as aglycones, glycosides and methylated derivatives.

For example, flavonoids occur as aglycones, glycosides, and methylated derivatives. With reference to Formula II, the basic flavonoid structure is aglycone. Six-member ring condensed with the benzene ring is either a α-pyrone (flavonols and flavanones) or its dihydroderivative (flavonols and flavanones). The position of the benzenoid substituent divides the flavonoid class into flavonoids (2-position) and isoflavonoids (3-position). Flavonols differ from flavanones by hydroxyl group at the 3-position and a C2-C3 double bond (Narayana, et al., “Bioflavonoids classification, pharmacological, biochemical effects and therapeutic potential,” Indian Journal of Pharmacology, vol. 33, no. 1, pp. 2-16, (2001)). Flavonoids are often hydroxylated in positions 3, 5, 7, 2, 3′, 4′, and 5′. Methyl ethers and acetyl esters of the alcohol group are known to occur in nature. When glycosides are formed, the glycosidic linkage is normally located in positions 3 or 7 and the carbohydrate can be L-rhamnose, D-glucose, glucorhamnose, galactose, or arabinose (Middleton, “The flavonoids,” Trends in Pharmacological Sciences, vol. 5, pp. 335-338, (1984)).

Flavonoids are plant secondary metabolites and are found in several parts of the plant (e.g., fruits, vegetables, grains, bark, roots, stems, flowers). Flavonoids are used by vegetables for their growth and defense against plaques. Flavonoids protect plants from different biotic and abiotic stresses and act as unique UV filters, function as signal molecules, allopathic compounds, phytoalexins, detoxifying agents and antimicrobial defensive compounds (Pance A N., et al., J. Nutr. Sci., 5: e47. 2016). Flavonoids are considered as an indispensable component in a variety of nutraceutical, pharmaceutical, medicinal and cosmetic applications. This is attributed to their anti-oxidative, anti-inflammatory, anti-mutagenic and anti-carcinogenic properties coupled with their capacity to modulate key cellular enzyme function.

For example, Acetylcholinesterase (AChE) is a key enzyme in the central nervous system and inhibition of it leads to increases of neural acetylcholine levels. A number of flavonoids have been reported for their anti-cholinesterase activity. Studies showed that quercetin and macluraxanthone possess a concentration-dependent inhibition ability against AChE and butyrylcholinesterase (BChE) (Pance A N., et al., J. Nutr. Sci., 5: e47. 2016; Khan M T., et al., Chem Biol Interact 181, 383-389. 2009).

Flavonoids have capacity to act as antioxidants. Flavonoids can prevent injury caused by free radicals in various ways and one way is the direct scavenging of free radicals. Flavonoids are oxidized by radicals, resulting in a more stable, less-reactive radical. In other words, flavonoids stabilize the reactive oxygen species by reacting with the reactive compound of the radical. Because of the high reactivity of the hydroxyl group of the flavonoids, radicals are made inactive. In some embodiments, flavonoids induce or enhance expression and/or activity of one or more components of antioxidant pathways (e.g., Nrf2 pathway, AP-1 pathway) (Farooqui, T. Front. Genet., 5:60. 2014). In some embodiments, the disclosed compositions and methods take advantage of the antioxidant and other (e.g., anti-inflammatory, anti-mutagenic, and anti-carcinogenic properties coupled with their capacity to modulate key cellular enzyme functions) properties of flavonoids.

As introduced above, based on their structures, flavonoids can be divided into several subclasses including, flavones, flavanones, flavonols, isoflavones, anthocyanins, flavanols (catechins), chalcones, neoflavonoids. Accordingly, in preferred embodiments, the disclosed compositions can contain one or more flavones, flavanones, flavonols, isoflavones, anthocyanins, flavanols (catechins), chalcones, neoflavonoids, or combinations thereof.

Core structures for flavonoid subclasses include:

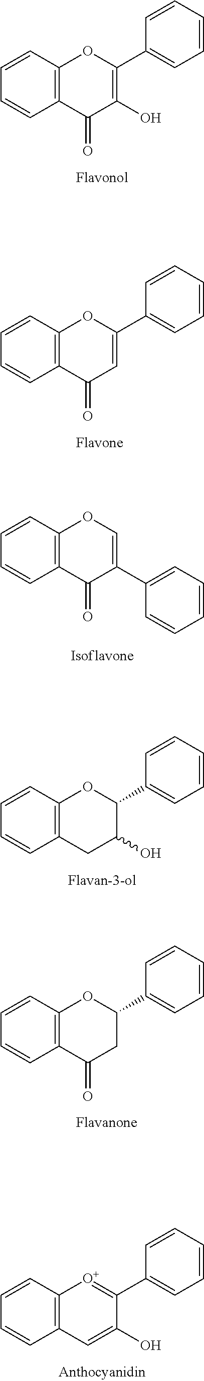 [SMILES: O=c1c(O)c(-c2ccccc2)oc2ccccc12 ; O=c1cc(-c2ccccc2)oc2ccccc12 ; O=c1c(-c2ccccc2)coc2ccccc12 ; OC1Cc2ccccc2O[C@@H]1c1ccccc1 ; O=C1C[C@@H](c2ccccc2)Oc2ccccc21 ; Oc1cc2ccccc2[o+]c1-c1ccccc1]

See, e.g., Nishiumi, et al., “Dietary flavonoids as cancer-preventive and therapeutic biofactors, Front Biosci (Schol Ed). 3:1332-62 (2011). doi: 10.2741/229, and Kumar and Pandey, Chemistry and Biological Activities of Flavonoids: An Overview, Volume 2013 IArticle ID 162750 I, 16 pages, doi.org/10.1155/2013/162750, each of which is specifically incorporated by reference herein in its entirety.

Flavones are widely present in leaves, flowers and fruits as glucosides. Celery, parsley, red peppers, chamomile, mint and Ginkgo biloba are among the major sources of flavones. Exemplary flavones include luteolin, apigenin and tangeritin.

Flavanones, also called dihydroflavones, have the C ring saturated; the A- and B-rings can be substituted by sugar or methyl groups. Flavanones are generally present in all citrus fruits such as oranges, lemons and grapes. The flavanones account for approximately 95% of the total flavonoids in the citrus. Citrus flavanones are typically present in the glycoside or aglycone forms. Hesperitin, naringenin and eriodictyol are examples of flavanones.

Flavonols are a class of flavonoids characterized by the presence of a 3-hydroxyflavone backbone (3-hydroxy-2-phenylchromen-4-one (IUPAC)). Onions, kale, lettuce, tomatoes, apples, grapes and berries are rich sources of flavonols. Apart from fruits and vegetables, tea and red wine are also sources of flavonols. Flavonols are very diverse in methylation and hydroxylation patterns which gives rise to their diversity. Exemplary flavonols include quercetin, rutin, myricetin, kaempferol, fisetin, morin, and isorhamnetin. In some embodiments, the disclosed compositions contain one or more flavonols such as quercetin, rutin, myricetin, kaempferol, fisetin, morin, isorhamnetin, or derivatives or variants thereof. The term “derivative” does not mean that the derivative is necessarily synthesized from the parent compound either as a starting material or intermediate, although this may be the case. The term “derivative” can include salts (for example, pharmaceutically acceptable salts), prodrugs, or metabolites of the parent compound. In preferred embodiments, the phenolic compound(s) is one or more flavonols. In particularly preferred embodiments, the phenolic compound(s) is rutin, quercetin, and/or a derivative thereof such as hyperoside, quercitrin, 3-O-methylquercetin, quercetin 3′-O-Sulfate or any others in Table 1 (e.g., isoquercetin, rutin, quercetin, quercitrin, hyperoside, quercimeritrin, baimaside, querciturone, 3-O-methylquercetin, quercetin 3-sambubioside, miquelianin, spiraeoside, isorhamnetin, quercetin 3′-O-sulfate, quercetin 7-O-glucoside, quercetin 3-O-Rhamnoside, quercetin 3,4′-diglucoside, and quercetin 3-sophorotrioside).

TABLE 1
Structural information of Quercetin and derivatives thereof.

| Name of | | |
|---|---|---|
| variant | Structure | Pharmacological activity |
| Isoquercetin | 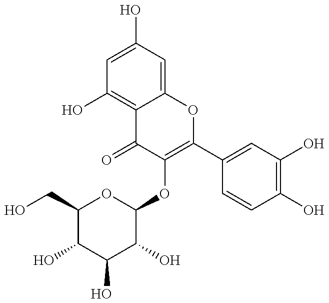 [SMILES: O=c1c(O[C@@H]2O[C@H](CO)[C@@H](O)[C@H](O)[C@H]2O)c(-c2ccc(O)c(O)c2)oc2cc(O)cc(O)c12] | Antibacterial, anticoagulant, antihistamine, anti-inflammatory, antispasmodic, antioxidant, hepatoprotective, monoamine oxidase inhibitor, pesticide, tyrosinase inhibitor, quinone reductase inducer. |
| Rutin | 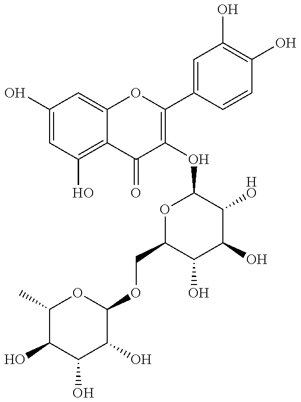 [SMILES: C[C@@H]1O[C@@H](OC[C@H]2O[C@@H](Oc3c(-c4ccc(O)c(O)c4)oc4cc(O)cc(O)c4c3=O)[C@H](O)[C@@H](O)[C@@H]2O)[C@H](O)[C@H](O)[C@H]1O] | Antioxidant anti-inflammatory anti-allergy, antitumor activity. |
| Quercetin | 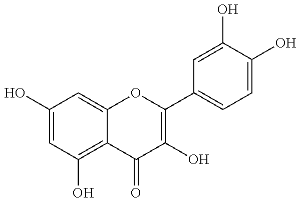 [SMILES: O=c1c(O)c(-c2ccc(O)c(O)c2)oc2cc(O)cc(O)c12] | Antioxidant activity Neurological effects Antivaral activity Anticancer activity Cardiovascular protection Antimicrobial activity Anti-inflammatory activity Hepatoprotective activity |
| Quercitrin | 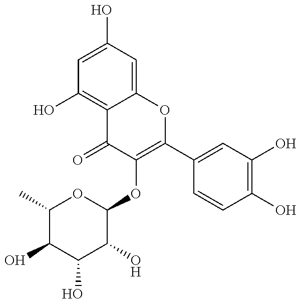 [SMILES: C[C@@H]1O[C@@H](Oc2c(-c3ccc(O)c(O)c3)oc3cc(O)cc(O)c3c2=O)[C@H](O)[C@H](O)[C@H]1O] | Anti-inflammatory Anti-proliferative Pro-apoptotic in carcinoma Inhibitor of osteoclastic differentiation |
| Hyperoside | 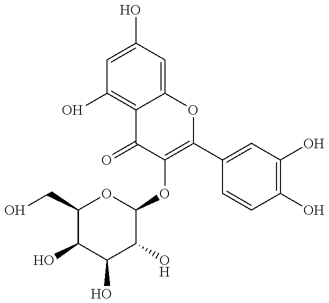 [SMILES: O=c1c(O[C@@H]2O[C@H](CO)[C@H](O)[C@H](O)[C@H]2O)c(-c2ccc(O)c(O)c2)oc2cc(O)cc(O)c12] | Anti-inflammatory Antidepressant Neuroprotective Cardioprotective Anti-diabetic Anti-cancer Anti-fungal Gastroprotective Antioxidant |
| Quercimeritrin | 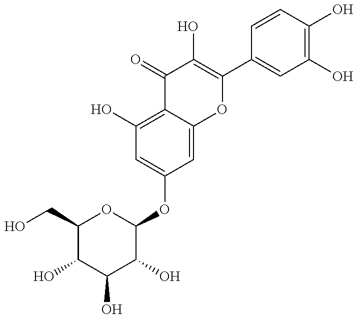 [SMILES: O=c1c(O)c(-c2ccc(O)c(O)c2)oc2cc(O[C@@H]3O[C@H](CO)[C@@H](O)[C@H](O)[C@H]3O)cc(O)c12] | Antimicrobial Anti-cancer |
| Balmaside | 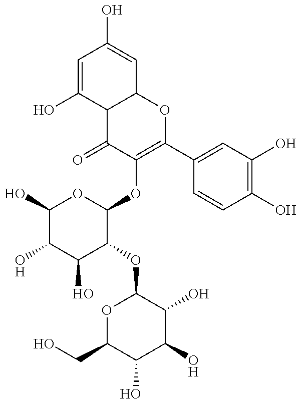 [SMILES: O=C1C(O[C@@H]2O[C@H](O)[C@@H](O)[C@H](O)[C@H]2O[C@@H]2O[C@H](CO)[C@@H](O)[C@H](O)[C@H]2O)=C(c2ccc(O)c(O)c2)OC2C=C(O)C=C(O)C12] | Cytoprotective Antioxidant |
| Querciturone | 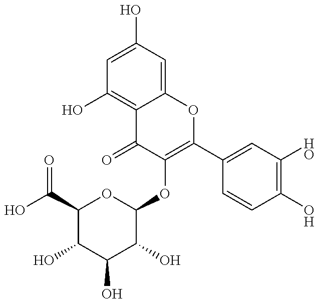 [SMILES: O=C(O)[C@H]1O[C@@H](Oc2c(-c3ccc(O)c(O)c3)oc3cc(O)cc(O)c3c2=O)[C@H](O)[C@@H](O)[C@@H]1O] | Antioxidant Anti-inflammatory Angiotensin converting enzyme inhibitor |
| 3-O- Methylquercetin | 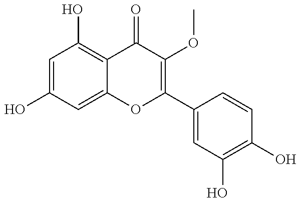 [SMILES: COc1c(-c2ccc(O)c(O)c2)oc2cc(O)cc(O)c2c1=O] | Antibacterial, anticoagulant, antihistamine, anti-inflammatory, antispasmodic, antioxidant, hepatoprotective, monoamine oxidase inhibitor, pesticide, tyrosinase inhibitor, quinone reductase inducer. |
| Quercetin 3- sambubioside | 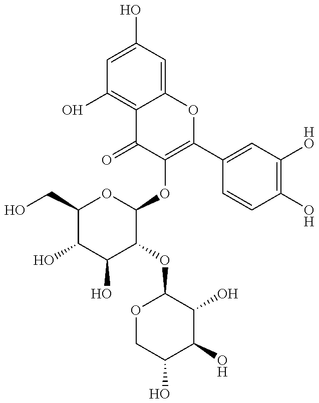 [SMILES: O=c1c(O[C@@H]2O[C@H](CO)[C@@H](O)[C@H](O)[C@H]2O[C@@H]2OC[C@@H](O)[C@H](O)[C@H]2O)c(-c2ccc(O)c(O)c2)oc2cc(O)cc(O)c12] | Antioxidant anti-inflammatory anti-allergy, antitumor activity. |
| Miquelianin | 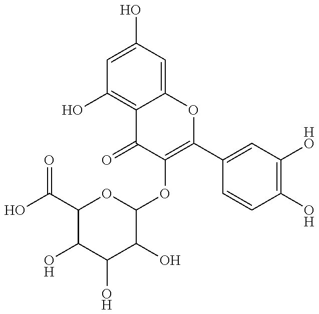 [SMILES: O=C(O)C1OC(Oc2c(-c3ccc(O)c(O)c3)oc3cc(O)cc(O)c3c2=O)C(O)C(O)C1O] | Antioxiddant activity Neurological effects Antiviral activity Anticancer activity Cardiovascular protection Antimicrobial activity Anti-inflammatory activity Hepatoprotective activity |
| Spiraeoside | 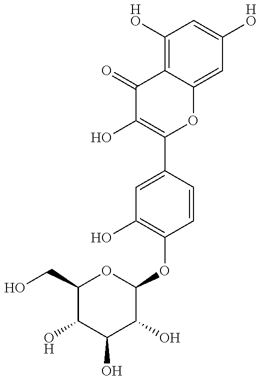 [SMILES: O=c1c(O)c(-c2ccc(O[C@@H]3O[C@H](CO)[C@@H](O)[C@H](O)[C@H]3O)c(O)c2)oc2cc(O)cc(O)c12] | Anti-inflammatory Anti-proliferative Pro-apoptotic in carcinoma Inhibitor of osteoclastic differentiation |
| Isorhamnetin | 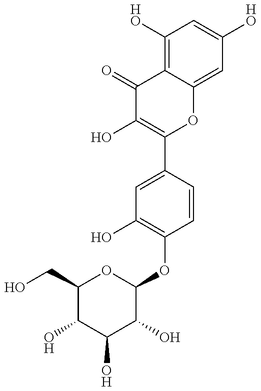 [SMILES: O=c1c(O)c(-c2ccc(O[C@@H]3O[C@H](CO)[C@@H](O)[C@H](O)[C@H]3O)c(O)c2)oc2cc(O)cc(O)c12] | Antibacterial, anticoagulant, antihistamine, anti-inflammatory, antispasmodic, antioxidant, hepatoprotective, monoamine oxidase inhibitor, pesticide, tyrosinase inhibitor, quinone reductase inducer. |
| Quercetin 3′-O- sulfate | 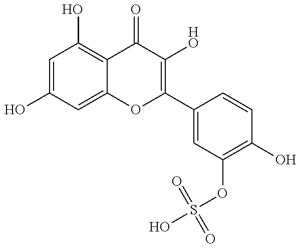 [SMILES: O=c1c(O)c(-c2ccc(O)c(OS(=O)(=O)O)c2)oc2cc(O)cc(O)c12] | Lipoprotein oxidation inhibitor |
| Quercetin 7-O- glucoside | 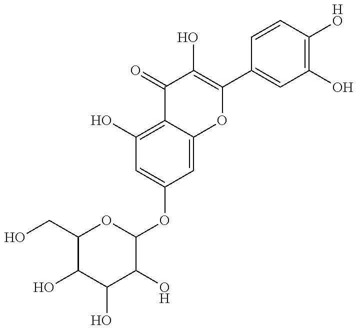 [SMILES: O=c1c(O)c(-c2ccc(O)c(O)c2)oc2cc(OC3OC(CO)C(O)C(O)C3O)cc(O)c12] | Antioxidant activity against peroxyl and hydroxyl radicals |
| Quercetin3-O- rhamnoside | 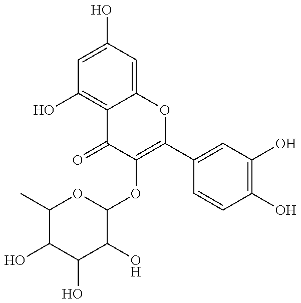 [SMILES: CC1OC(Oc2c(-c3ccc(O)c(O)c3)oc3cc(O)cc(O)c3c2=O)C(O)C(O)C1O] | Antimicrobial activity Antitumoral activity |
| Quercetin 3,4′- diglucoside | 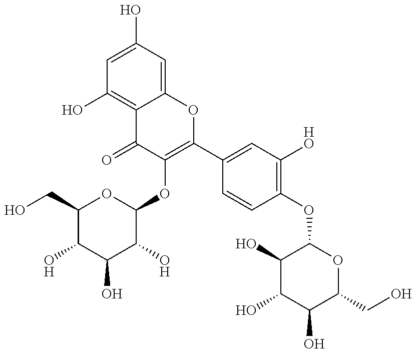 [SMILES: O=c1c(O[C@@H]2O[C@H](CO)[C@@H](O)[C@H](O)[C@H]2O)c(-c2ccc(O[C@@H]3O[C@H](CO)[C@@H](O)[C@H](O)[C@H]3O)c(O)c2)oc2cc(O)cc(O)c12] | Antioxidant activity |
| Quercetin 3- sophorotrioside | 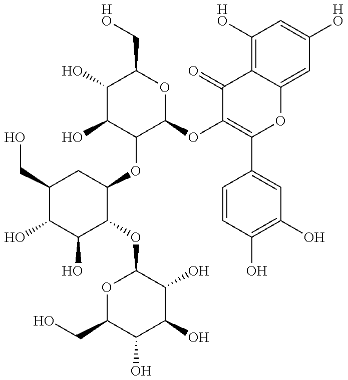 [SMILES: O=c1c(O[C@@H]2O[C@H](CO)[C@@H](O)[C@H](O)C2O[C@@H]2C[C@H](CO)[C@@H](O)[C@H](O)[C@H]2O[C@@H]2O[C@H](CO)[C@@H](O)[C@H](O)[C@H]2O)c(-c2ccc(O)c(O)c2)oc2cc(O)cc(O)c12] | Antibacterial anticoagulant, antihistamine, anti-inflammatory, antispasmodic, antioxidant, hepatoprotective, monoamine oxidase inhibitor, tyrosinase inhibitor, quinone reductase inducer. |

Isoflavones are a large and very distinctive subgroup of flavonoids. Their distribution in plants is limited compared to other flavonoids, and they are found predominantly in legumes, particularly, soybeans, chickpea, peanuts and alfalfa. Exemplary isoflavones include glycitein, genistein and daidzein.

Anthocyanins are water-soluble plant pigments responsible for the blue, purple, and red color of many plant tissues. They occur predominantly in the outer cell layers of various fruits such as cranberries, black currants, red grapes, merlot grapes, raspberries, strawberries, blueberries, bilberries and blackberries. The color of the anthocyanin depends on the pH and also by methylation or acylation at the hydroxyl groups on the A and B rings. Exemplary anthocyanins include cyanidin, delphinidin, malvidin, petunidin, pelargonidin and peonidin

Flavanols also called dihydroflavonols or catechins, are the 3-hydroxy derivatives of flavanones. They are a highly diversified and multisubstituted subgroup. Flavanols are also referred to flavan-3-ols as the hydroxyl group is always bound to position 3 of the C ring. Flavanols are found abundantly in bananas, apples, blueberries, peaches and pears. Non-limiting examples of flavanols include (+)-catechin, (−)-epicatechin (EC), (−)-epicatechin gallate (ECG), (−)-epigallocatechin (EGC) and (−)-epigallocatechin gallate (EGCG).

Chalcones are characterized by the absence of ‘ring C’ of the basic flavonoid skeleton structure and are also referred to as open-chain flavonoids. Examples of chalcones include phloridzin, arbutin, phloretin and chalconaringenin. Chalcones occur in significant amounts in tomatoes, pears, strawberries, bearberries and certain wheat products.

Neoflavonoids have a 4-phenylchromen backbone with no hydroxyl group substitution at position 2. An exemplary neoflavonoid is calophyllolide, which can be found in Calophyllum inophyllum seeds and the bark and timber of the Mesua thwaitesii plant.

In other embodiment the phenolic compound is p-coumaric acid, having the structure:

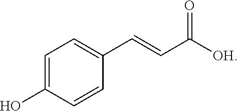 [SMILES: O=C(O)/C=C/c1ccc(O)cc1]

In some embodiments, the disclosed compositions can contain, and/or methods can utilize, a plurality of two, three, four or more different phenolic compounds, such as flavonols including quercetin and rutin and kaempferol, and/or other phenolic compounds such as p-coumaric acid. One of ordinary skill in the art can readily determine the appropriate ratios (e.g., molar or mass) of the phenolic compounds in such compositions. It is to be understood that in some embodiments, having a plurality of phenolic compounds may confer a synergistic effect (e.g., the effect of the combination is higher than the sum of the effect of each phenolic compound alone). In specific embodiments, quercetin and rutin are together in the same or different formulations at a quercetin:rutin ratio of 1:1, 0.75:25, or 0.25:0.75.

B. Food, Feed Additives and Supplements

The disclosed compositions containing one or more phenolic compounds can be used independently (e.g., as a food or supplement) and/or in combination with other animal feed (e.g., commercially available feeds). Thus, in some embodiments, the disclosed compositions can be used as a food, feed additive or supplement, or nutraceutical.

For example, in some embodiments, compositions containing one or more phenolic compounds can be directly administered to a subject (e.g., insect pollinator) by ingestion (e.g., diluted in water or other suitable solvent, or as a powder). However, in one alternative embodiment, the subject may be caused to ingest a composition containing one or more phenolic compounds by providing the composition to the subject simultaneously, separately or sequentially with typical feeding. The subject may be caused to ingest the one or more phenolic compounds by providing the one or more phenolic compounds by dietary means, such as in or mixed with an animal feed, as a dietary supplement, and/or in a drinking water. It should be noted that, dependent on the solubility of the one or more phenolic compounds used, it may be beneficial to introduce a co-solvent to solubilize or aid dissolution in water at an effective concentration.

Feed compositions are well known in the art for various species of animals, such as insect pollinators including honeybees. Honeybees, like all other animals, require essential nutrients for survival and reproduction. Honeybees require carbohydrates (e.g., sugars in nectar or honey), amino acids (e.g., protein from pollen), lipids (e.g., fatty acids, sterols), vitamins, minerals (e.g., salts) and water. Additionally, these nutrients must be present in the right ratios for honeybees to survive and thrive. Honeybees and other bee species collect pollen from flowers to obtain protein, fats, sterols, vitamins, and minerals. They also collect floral nectar, aphid honeydew, and other sugar sources like tree sap to provide them with carbohydrates. Both adult and larval stages depend on pollen and nectar for nourishment. Typically, bees rely on natural forage to provide floral nectar and pollen to supply the nutrition of their colonies.

However, in some cases bees may be fed by other sources. For example, a common way to feed honeybees during periods when they cannot obtain sufficient pollen is to add pollen substitutes to the colony in the form of a patty or a solution. These substitutes are typically composed of plant (e.g., soy or corn gluten) and/or animal proteins (e.g., whole eggs), oils, and brewer's yeast.

Currently known artificial diets for honeybees include liquid artificial nectars that typically include a carbohydrate or sugar source, pollen patties made of pollen and sugars, patties made of soy protein (usually solvent extracted) mixed with brewers yeast and sugar, patties made from a mixture of soy flour, Torula or brewers yeast, pollard, vegetable oil, vitamin mix and irradiated honey or malt, patties made from a mixture of expeller-pressed soy flour, pollard, cotton seed oil, vitamin mix and irradiated honey or malt, and Haydak diet patties made of soy meal, brewers yeast, sugar, and powered skim milk. In some embodiments, any of the aforementioned diets may be supplemented with the compositions containing an effective amount of one or more phenolic compounds before administration to the bees.

Nectar is the main source of carbohydrates for honeybees. The amount of nectar needed per colony depends on how concentrated the sugars are in the nectar. A worker bee needs 11 milligrams (mg) of dry sugar each day. Supplemental sugar in the form of honey or as a sugar syrup can be provided to honeybees as necessary. Typically, the sugar syrup contains a 1:1 or 2:1 sugar to water ratio (measured by volume or by weight) but ratios may vary depending upon colony or individual requirements. In some embodiments, the sugar syrup or honey is supplemented with the compositions containing an effective amount of one or more phenolic compounds before administration to the bees.

In some embodiments, the compositions containing one or more phenolic compounds may be mixed or otherwise combined with any source of nutrition for the insect pollinator (e.g., honeybee, bumblebee). Such sources include a source of carbohydrates, proteins, lipids, vitamins, minerals, water and combinations thereof. Exemplary sources include natural or artificial nectar, honey, sugar, sugar syrup, pollen or pollen substitute, soy flour, soy meal, gluten (e.g., soy or corn), skim milk, yeast, pollard, oil, and combinations thereof. For example, pollen can be purchased from commercial vendors or can be trapped from bees. In some embodiments, the sources can be, or can be derived from, commercially available bee feed such as AP23©, BEE-PRO®, FEEDBEE, MEGABEE and ULTRA BEE. In preferred embodiments, any of the foregoing may be mixed or otherwise combined with a composition containing one or more phenolic compounds to form a final composition (e.g., food, feed additive or supplement, or nutraceutical) that contains an effective amount of the one or more phenolic compounds.

The one or more phenolic compounds may be included in an animal feed, or in an animal feed supplement or premix, for the feed of commercial pollinators such as honeybees and bumblebees. In one embodiment, the one or more phenolic compounds may be included in, or used to supplement, a pollen substitute, which can be a complete feed. A complete feed is designed to contain all the protein, carbohydrates, vitamins, lipids, minerals, and other nutrients necessary for proper growth, reproduction and health of the pollinator.

C. Feed Formulations

An animal feed or feed supplement for use in the methods described herein may include one or more phenolic compounds, proteins, lipids, carbohydrates, minerals, water, other nutrients or ingredients, or combinations thereof.

Protein and lipid sources can be corn gluten sources in combination with soy concentrate, barley flour, yeast, and/or corn distillers dry grains, soy sources and egg or egg product sources. The egg source is also a good source of cholesterol. Soy contributes one of the most complete profiles of essential amino acids of any plant material, and it is also a source of lipoproteins, which help deliver sterols and polyunsaturated fatty acids. It is a naturally lipid-rich food material. Examples of soy sources include soy flour (e.g., expeller pressed, solvent extracted), soy meal, soy milk, and suspended soy extract. Eggs, especially egg yolks, are sources of extremely high amounts of proteins that are a standard of nutritional completeness. Eggs are also a rich source of lipids, including cholesterol which, as a sterol, is an essential nutrient for honey bees. The lipids in egg yolk are also rich in polar components such as lecithin, which is highly digestible, nutritious, and a natural emulsifier. Eggs also contribute texture by increasing viscosity. Eggs are a complete source of B vitamins, vitamin A (in a complex of carotenoids), and vitamin E, and are also a fairly complete source of minerals. Also, vegetable oils can be used as a lipid source. Examples include soy oil, safflower oil, corn oil, peanut oil, sunflower oil, canola oil, rapeseed oil, cottonseed oil, and flax oil.

The carbohydrate (e.g., sugar) source in the formulations can serve as a feeding stimulant, a source of carbon for building blocks for growth, a source of energy, a viscosity increasing agent (texturizer), and a humectant (water retaining agent), which lower water activity that reduces microbial growth and inhibits chemical reactions that help deteriorate diets. Examples of sugar sources include sucrose, e.g., crystalline or granulated sugar; other crystalline or granulated sugars, e.g., fructose, glucose (also denoted as dextrose) or maltose; high fructose corn syrup, e.g., HFCS55, or other sugar syrup. They can be used in the solid form or as a syrup.

Anti-fungal and/or anti-microbial agents are optionally added to the formulations to prevent premature deterioration of the formulations. These are desirable because at typical hive temperatures, microbes can proliferate rapidly and spoil the feeds and serve as potential pathogens to the bees. Exemplary antimicrobial agents are sorbic acid and its salts, propionic acid and its salts, the series of parabens (methyl, ethyl, propyl, and butyl form), and benzoic acid and its salts. Potassium sorbate, a fatty acid, has well-demonstrated anti-fungal and anti-microbial properties. Sodium propionate is an effective anti-microbial agent and approved preservative. Other anti-fungal and/or antimicrobial agents are known in the art, including for example calcium propionate.

The compositions can be formulated in any form suitable for administration (e.g., ingestion) to the insect pollinator. In some embodiments, the compositions can be in the form of a liquid (e.g., sugar syrup, high-fructose corn syrup). A liquid formulation can be a solution, suspension or emulsion. In some embodiments, liquid formulations provide an evenly mixed, water-dispersible, substantially homogeneous, substantially non-clumping, pourable, or flowable liquid wherein nutrients are dissolved, suspended, and/or emulsified therein.

In some embodiments, the compositions are formulated as a solid (e.g., a powder, patty, candy). The dry formulations may be directly administered to insect pollinators such as bees without liquid being added. In some embodiments, the compositions may be formulated in dry form, which may subsequently be mixed with water or a sugar syrup prior to use to form a liquid. A patty or a semi-dry formulation may be formed by using less syrup or a solid carbohydrate source and bringing the product to a dough like or powdery consistency, e.g., that can be placed in or near a colony for feeding.

Formulations of the various compositions can be pH balanced, e.g., to mimic the pH of natural pollen and promote a healthy gut environment in the pollinator. Accordingly, the formulation can include one or more organic acids or phosphoric acid as acidifier sources to achieve the desired pH (e.g., in the range of 3.5 to 7). The preferred pH is about 4.5. Exemplary acidifier compounds are the organic acids such as citric acid, acetic acid, lactic acid, malic acid, fumaric acid, or succinic acid and combinations of organic acids such as malic acid, fumaric acid, and pyruvic acid.

In some embodiments, the particle size of the formulation is controlled to mimic the particle size of natural pollen. For example, in some embodiments, the particle size of the formulation is about 35 microns or smaller. Optionally, anti-microbial agents may be included to prevent pre-mature deterioration of the formulations.

### III. Methods

A. Methods of Use

Methods of using the disclosed compositions are provided. The compositions provided herein such as a phenolic compound, or a composition including a phenolic compound, can be used as a food, feed additive or supplement, or nutraceutical. Such foods, feed additives or supplements, or nutraceuticals are useful for prophylactically protecting insect pollinators against the negative effects of neurotoxic pesticides (e.g., by administration before exposure to a pesticide). In some embodiments, the disclosed compositions including foods, feed additives, feed supplements, or nutraceuticals are useful for treating insect pollinators against the negative effects of neurotoxic pesticides (e.g., by administration during and/or after exposure to a pesticide).

Methods of protecting a cognitive function of an insect pollinator by administering an effective amount of any of the disclosed compositions such as a phenolic compound, or a composition including a phenolic compound, to an insect pollinator in need thereof are provided. For example, in some embodiments, the methods include protecting the learning and/or memory capabilities of an insect pollinator by administering an effective amount any of the disclosed compositions to the insect pollinator.

Methods for preventing or reducing impairment of cognitive function in an insect pollinator by administering to the insect pollinator a composition (e.g., a food, feed additive or supplement, or nutraceutical) containing an effective amount of one or more phenolic compounds to prevent or reduce impairment of a cognitive function in the insect pollinator are also provided. Preferably, the cognitive function includes but is not limited to, learning, memory, attention, decision-making and other forms of information acquisition, storage, processing, retrieval and use, navigation, motor activity, sucrose sensitivity, and combinations thereof.

Thus, in some embodiments, the methods reduce, diminish, or prevent a reduction or loss in memory, learning, navigation skills, motor activity, or combination thereof in an insect pollinator upon exposure to a pesticide, by administering any of the disclosed compositions.

Methods of controlling insect pests are also provided. In some embodiments, a method of controlling insect pests involves treating an area with a pesticide and administering to the insect pollinator a composition including an effective amount of one or more phenolic compounds to prevent or reduce impairment of a cognitive function and/or increase a cognitive function in the insect pollinator. The area can be a geographical space (e.g., a field) or part of an organism (e.g., a plant). In some embodiments, the area is occupied by insect pests and insect pollinators. The area can be treated with the pesticide at any suitable time, e.g., before or after the composition is administered to the insect pollinators. In some embodiments, the area is treated with the pesticide by foliar application, soil injection, tree injection, ground application as a granular or liquid formulation, or as a pesticide-coated seed treatment.

Preferably, the area is treated with an effective amount of pesticide to reduce the number of insect pests in the area. For example, the pesticide can be lethal to insect pests and/or in an amount that is sublethal to insect pollinators. Exemplary insect pests that can be targeted include aphids, thrips, whiteflies, mites, leafhoppers, mealybugs, spittlebugs, fleas, termites, scales (e.g., armored scales, soft scales), and beetles.

Typically, the disclosed methods prevent, reduce, decrease, or inhibit one or more adverse effects associated with pesticide exposure. For example, in some embodiments, the disclosed methods reduce, decrease, or inhibit one or more mechanisms of mitochondrial dysfunction (e.g., reduced electron transport, reduced ATP production, altered mitochondrial membrane potential), e.g., in the brain of an insect pollinator by administering any of the disclosed compositions. In some embodiments, the disclosed methods induce or enhance expression and/or activity of one or more components of antioxidant and overall detox pathways (e.g., Nrf2 pathway, AP-1 pathway, CYP450 proteins) in the brain or other organs of the insect pollinator by administering any of the disclosed compositions to the insect pollinator.

In some embodiments, the methods may enhance cognitive function. For example, methods for enhancing cognitive function (e.g., learning, memory, speed and accuracy of decisions) in an insect pollinator by administering to the pollinator a disclosed composition containing an effective amount of one or more phenolic compounds are provided.

B. Methods of Administration

The disclosed compositions and formulations can be administered to the subject in need thereof (e.g., an insect pollinator) by a variety of suitable means. For example, the compositions and formulations can be ingested by the recipient insect pollinator.

Typically, the methods provide for administration of the disclosed compositions and formulations via ingestion through the normal feeds and feeding schedule of the insect pollinator. The provided compositions and formulations can be provided as food, feed additive or supplement, or nutraceutical. The compositions and formulations can be placed in an area where bees are located or within feeding vicinity of bees, such as in or adjacent to a bee hive or bee cage, or also inside the hive as a patty or as a liquid. By “patty” is meant a mixture of sugar syrup and a bee diet formulation to form soft pliable dough-like consistency that is pressed into a thin patty. Patties are typically provided to honey bee colonies to support the protein and nutritional need of the colony.

By way of example, mechanisms for providing the disclosed compositions and formulations to bees are summarized below. However, these are not intended to be limiting. One of skill in the art can readily determine appropriate methods for administration of the composition to any insect pollinator of interest. Appropriates amounts and timing of feeding for various organisms are known to those skilled in the art and are readily ascertainable.

The compositions and formulations may be fed to bees or a colony of bees in a variety of ways. For example, the compositions may be formulated as a liquid and fed within a hive in a horizontal feeder in place of a comb. Alternatively, or in addition, the composition may be placed in a vertical feeder, which is in turn placed on top of a comb within the hive.

The compositions may be formulated as a liquid, a patty or a biscuit. In certain embodiments, the composition is provided adjacent to a comb in the hive, e.g., on top of the comb. The compositions can be provided on a mesh through which the bees can pass. In certain embodiments, the compositions formulated as a liquid is provided in an inverted jar inserted into a hole in the roof of a hive. In certain embodiments, the compositions may be provided in an area surrounding a hive (e.g., within an apiary). Thus, the bees may eat the composition as part of their normal foraging. In such embodiments, the composition may be formulated as a liquid or a powder.

In some embodiments, the composition may for example be provided via a frame feeder, or may be poured or sprayed.

In any of the foregoing, the compositions and formulations can be provided to the insect pollinator before or after exposure to one or more pesticides. This can be over any period of time, for example, minutes, hours, days, or weeks. In some embodiments the compositions and formulations are provided to the insect pollinator one or more times between about 1 hour and about 10 days after pesticide exposure.

Additionally, or alternatively, in preferred embodiments, the compositions and formulations can be provided to the insect pollinator before exposure to one or more pesticides. For example, the compositions and formulations can be provided in the range of between about 1 hour and about 10 days before exposure to one or more pesticides.

Typically, the compositions and formulations are provided 1-3 days (e.g., 1, 2, or 3 days) before exposure to one or more pesticides. For example, migratory bee keepers who use their hives to pollinate almond, apple, plum and other orchards can provide the compositions and formulations to their colonies before (e.g., 1, 2, or 3 days) releasing them to the open for foraging.

When administration is via ingestion, the compositions and formulations are typically provided to the insect pollinators ad lib, i.e., the pollinator feeds freely as desired. For example, the compositions containing an effective amount of one or more phenolic compounds can be formulated as a liquid (e.g., sugar syrup) that is placed in a bag or jar in, on, or near the hive. The bees can access and ingest the composition as desired over the period of time before exposure to a pesticide. For example, the bees can be fed with the composition ad lib over a period of 1-10 days before exposure to one or more pesticides.

The pollinator may ingest an effective amount of one or more of the phenolic compounds on a single, repeated, or regular basis. For example, the pollinator may ingest an effective amount of one or more phenolic compounds one, two, three, or more times weekly, every other day, every day, or more than once every day (e.g., once, twice, three, or more times every day) during the performance of the disclosed methods or uses. The Examples below illustrate that in some embodiments, a protective effect may last more than 36 hours after the last dose.

In some embodiments, the one or more phenolic compounds are included in a feed, a feed supplement, and/or in drinking water and the pollinator ingests the one or more phenolic compounds when they eat and/or drink, and optionally every time they eat and/or drink. This ingestion of an effective amount of one or more phenolic compounds may continue through a period of time of the animal's lifespan that may correspond to a period of time that is, is up to, or is at least, 5%, 10%, 25%, 30%, 40%, 50%, 60%, 70%, 80%, 90%, 95% or substantially 100% of the life of the animal from birth to death. The ingestion of an effective amount of one or more phenolic compounds may start on the day of the animal's birth, or at the age of 1, 2, 3, 4, 5, 6, 7, 8, 9, 10, 11, 12, 13, 14, 15, 16, 17, 18, 19, 20, 21, 22, 23, 24, 25, 26, 27 28, 29, 30, 31, 32, 33, 34, 35, 36, 37, 38, 39, 40, 41, 42, 43, 44, 45, 46, 47 days, or more. After the pollinator starts to ingest or absorb the one or compounds, it may continue to do so on a regular and repeated basis for a period of time that can be, or be up to, or at least, 1, 2, 3, 4, 5, 6, 7, 8, 9, 10, 11, 12, 13, 14, 15, 16, 17, 18, 19, 20, 21, 22, 23, 24, 25, 26, 27 28, 29, 30, 31, 32, 33, 34, 35, 36, 37, 38, 39, 40, 41, 42, 43, 44, 45, 46, 47 days, or more. Ingestion can also start while in larval stages, where adults can provide a brood with a single (mass provisioning species) or multiple (sequential provisioning) loads of supplemented food.

1. Effective Amounts

Because the administration of the compositions containing one or more phenolic compounds elicits a beneficial effect on one or more cognitive functions of the insect pollinator, the amount of the composition administered can be expressed as the amount effective to achieve a desired effect in the recipient pollinator. For example, in some embodiments, the amount of one or more phenolic compounds in the compositions is effective to enhance cognitive function (e.g., learning, memory, navigation) in an insect pollinator.

In some embodiments, the amount of one or more phenolic compounds in the compositions is effective to protect insect pollinators against the harmful cognitive effects of pesticides (e.g., neurotoxic pesticides). The amount of one or more phenolic compounds in the compositions can be effective to prevent reduction in memory, learning, navigation skills, motor activity, or combinations thereof in an insect pollinator upon exposure to a pesticide. The amount of one or more phenolic compounds in the compositions can be effective to reduce or diminish loss of memory, learning, navigation skills, motor activity, or combinations thereof in the insect pollinator upon exposure to a pesticide.

The amount of a composition administered to an insect pollinator is typically enough to prevent, reduce, decrease, or inhibit one or more adverse effects associated with pesticide exposure. For example, in some embodiments, the amount of one or more phenolic compounds in the compositions is effective to prevent, reduce, decrease, or inhibit one or more mechanisms of mitochondrial dysfunction (e.g., reduced electron transport, reduced ATP production, altered mitochondrial membrane potential). In other embodiments, the amount of one or more phenolic compounds in the compositions is effective to induce or enhance expression and/or activity of one or more components of antioxidant pathways (e.g., Nrf2 pathway, AP-1 pathway) in the brain or other organs of the insect pollinator.

Effective amounts can be expressed as total mass (e.g., mg), an amount per unit body weight of the recipient (e.g., mg/kg), as body surface-area based dosing (e.g., mg/m²) and the like. In some embodiments, the disclosed compositions can contain a phenolic compound in the concentration range of about 0.1 μM to about 100 mM, about 0.1 μM to about 10 mM, or about 0.1 μM to about 10 mM, or about 1 μM to about 10 mM, or 0.5 μM to about 5 mM, or about 0.1 μM to about 100 μM, or about 0.1 μM to about 10 μM, or about 1 μM to about 1 mM, or about 1 μM to about 100 μM, or any subrange between any of the foregoing. In some embodiments, the dosage is a specific dose between the foregoing ranges and subranges, inclusive of the end points. For example, in the experiments below, honeybees received 1 mM Rutin and 6 mM Quercetin. Bumblebees received 1 μM Rutin. The dosage of other tested phenolic compounds are also discussed in the Examples below, and such dosages can be utilized in the disclosed compositions and methods.

The actual effective amounts of an active agent (e.g., phenolic compound) can vary according to factors including the specific phenolic compound, the particular composition formulated, the mode of administration, and the weight or condition of the subject (e.g., insect pollinator) being administered, as well as the biological parameter (e.g., learning, memory, navigation) being evaluated or impacted.

Flavonoids exhibit a hormetic effect (i.e. low concentrations can often be better) and high concentrations may be toxic. Thus, typically a lower, but still effective amount, is preferred over of a higher amount. Preferably, the amount is nontoxic or at least sublethal for pollinators, particular honeybees and/or bumblebees.

2. Insect Pollinators

The disclosed compositions can be used to protect the cognitive function of a pollinator (e.g., an insect pollinator). For example, the compositions can be used for preventing or reducing impairment of cognitive function (e.g., learning, memory, navigation) in an insect pollinator. Pollination is the process by which a pollen grain moves from the anther (male part) of a flower to the stigma (female part). This is the first step in a process that produces seeds, fruits, and the next generation of plants. This can happen through self-pollination, wind and water pollination, or through the work of vectors (e.g., animals) that move pollen within the flower. Notably, it is estimated that between 75% and 95% of all flowering plants on the earth need help with pollination, i.e., they need pollinators (Ollerton J., et al., Oikos, 120:321-326. (2011)).

Many crops benefit from pollination by animals. Birds, bats, butterflies, moths, flies, beetles, wasps, small mammals, and bees are known pollinators. They visit flowers to drink nectar or feed off of pollen and transport pollen grains as they move from spot to spot. Crops that benefit from such pollinators include almond, apple, avocado, blueberry, canola, cantaloupe, cherry, blueberry, cranberry, cucumber, kiwifruit, nectarine, peach, pear, pepper, plum, prune, raspberry, squash (including pumpkin and gourd), strawberry, sunflower, and tomato; and also crops for seed production, such as alfalfa, asparagus, beet, cabbage and other crucifers, carrot, clover and onion.

In preferred embodiments, the pollinator is a managed species such as bees (for example bumblebees, honeybees, squash bees, mason bees, stingless bees, carpenter bees, etc.) but other wild bees (orchid bees, sweat bees). Additionally or alternatively, the pollinator includes or consists of one or more other insect pollinators (e.g. butterflies, moths, flies, beetles, wasps).

Honeybees and Bumblebees

In some embodiments, the insect pollinator is a member of the Apidae (which includes honeybees and bumblebees), Halictidae, Andrenidae, Megachilidae, or Colletidae families. In preferred embodiments, the insect pollinator is a member of the Apis genus within the Apidae family, such as but not limited to, A. mellifera, A. mellifera liguistica, A. mellifera carnica, A. mellifera caucasica, A. mellifera caucasica, A. mellifera iberiensis, A. mellifera scutellata, A. cerana, and A. dorsata.

In some embodiments, the insect pollinator is a bumblebee such as, but not limited to, buff-tailed bumblebee, cuckoo bumblebee, tree bumblebee, garden bumblebee, heath bumblebee, ruderal bumblebee, broken-belted bumblebee, white-tailed bumblebee, red-tailed bumblebee, early bumblebee, red-shanked bumblebee, and bilberry bumblebee. In some embodiments, the insect pollinator is a member of the Bombus genus within the Apidae family, such as but not limited to, Bombus terrestris, Bombus impatiens, Bombus occidentalis, Bombus atratus, Bombus vestalis, Bombus lucorum, Bombus bohemicus, Bombus hypnorum, Bombus sylvestris, Bombus hortorum, Bombus barbutellus, Bombus jonellus, Bombus ruderatus, Bombus soroeensis, Bombus lapidarius, Bombus rupestris, Bombus pratorum, Bombus ruderarius, Bombus monticola, Bombus pascuorum, Bombus campestris, Bombus humilis, Bombus muscorum, Bombus distinguendus, and Bombus sylvarum.

The disclosed compositions and methods can be used to limit the harmful effects of neurotoxic pesticides on any of the foregoing pollinators.

3. Pesticides

Typically, impairment of a cognitive function is induced by exposure to a pesticide, for example, a neurotoxic pesticide. Accordingly, the disclosed compositions may be useful to protect against pesticides that are neurotoxic. The disclosed compositions may be useful to protect against pesticides that can adversely affect cholinergic, GABAergic or glutamatergic neurotransmission. In some embodiments, the disclosed compositions may be useful to protect against pesticides that are toxic to mushroom bodies (main centers of learning and memory and integration in the insect brain) and the antennal lobes (major centers for primary integration of olfactory information). In some embodiments, the disclosed compositions may be useful to protect against pesticides that negatively affect mitochondrial function (e.g., electron transport, ATP production, mitochondrial membrane potential), increase apoptosis, increase oxidative stress, or combinations thereof in the brain, for example, the mushroom bodies of the insect pollinator.

Insecticides play an important role in the control of insect pests. Most of the chemical insecticides in use today are neurotoxic pesticides, and act by poisoning the nervous systems of the target organisms. The target sites for insecticides in insects are also found in mammals, hence insecticides vary in their levels of selectivity with regard to targets of toxicity, and mammals, including humans, may be sensitive to their toxicity. Insecticides have higher acute toxicity toward non-target species compared to other pesticides. There are several classes of insecticides that affect the pre- or post-synaptic terminals (e.g. pyrethroids, neonicotinoids, sulfomine derivatives, fipronil) as well as the dynamics occurring in the synaptic cleft (e.g. organophosphates and carbamates).

a. Neonicotinoids

In preferred embodiments, impairment of a cognitive function is induced by exposure to one or more pesticides that target (e.g., agonists of) the nicotinic acetylcholine receptor (nAChR). In preferred embodiments, impairment of a cognitive function is induced by exposure to one or more neonicotinoids. In preferred embodiments, methods of controlling insect pests involve treating an area with a pesticide, such as one or more neonicotinoids.

The introduction to the market in the early 1990s of imidacloprid and thiacloprid opened the neonicotinoid era of insect pest control. Neonicotinoids are now the most widely used pesticides in the world with a global market share of at least 26% of the insecticide market. Two of them-clothianidin and thiamethoxam-dominate the global market for insecticidal seed treatments and are used to coat the seeds of most of the annual crops planted around the world. In fact, more than 94% of the corn and more than 30% of the soy planted in the United States is pretreated with neonicotinoids. Acting systemically, this class of neurotoxic insecticides is taken up by plants, primarily through the roots, and translocates to all parts of the plant through xylemic and phloemic transport. Therefore, unlike older pesticides that evaporate or disperse shortly after application, neonicotinoid insecticides incorporate themselves into plant tissues, turning the plant itself into a tiny poison factory emitting toxin from its roots, leaves, stems, pollen, and nectar.

As the name suggests, neonicotinoids are similar in structure to nicotine and interact with the nicotinic acetylcholine receptors (nAChRs) of the insect central nervous system. Their capacity to cross the ion-impermeable barrier surrounding the central nervous system (BBB, blood-brain barrier) and their strong binding to nAChR in the bee's central nervous system are responsible for a unique chronic and sublethal toxicity profile.

Neonicotinoids mainly act agonistically on nAChRs on the post-synaptic membrane, mimicking the natural neurotransmitter acetylcholine by binding with high affinity (Van der Sluijs, J P., et al., Current Opinion in Environmental Sustainability, 5(3-4), 293-305. 2013). This induces a neuronal hyper-excitation, which can lead to the insect's death within minutes. Some of the major metabolites of neonicotinoids are equally neurotoxic, acting on the same receptors thereby prolonging the effectiveness as systemic insecticide. The nAChR binding sites in the vertebrate nervous system are different from those in insects, and in general they have lower numbers of nicotinic receptors with high affinity to neonicotinoids, which are the reasons that neonicotinoids show selective toxicity for insects over vertebrates (Van der Sluijs, J P., et al. 2013).

At their introduction, neonicotinoids were assumed to be more efficient than the organophosphates and carbamates that they replaced. As a seed treatment, they could be used in much lower quantities and they promised to be less polluting to the environment. It is however not the quantity that is relevant but the potency to cause harm, which results from toxicity, persistence and bioavailability to non-target species. Soon after the introduction of neonicotinoids, exposure to its residues in pollen, nectar, sowing dust etc., of non-target pollinating insects became clear.

Exemplary neonicotinoids include imidacloprid, thiacloprid, clothianidin, thiamethoxam, acetamiprid, nitenpyram, dinotefuran, and nithiazine. Many neonicotinoids are commercially available, for example as various insecticide formulations. Table 2 provides an exemplary list of neonicotinoids and their trade names.

TABLE 2
Exemplary list of commercial neonicotinoid containing products.

| Compound | Trade Name (Commercial Products) |
|---|---|
| Imidacloprid | ADMIRE ®PRO, Advantage ® or K9 Advantix ®, |
| | ENFORCE 75WSP, IMIDAPRO 4SC, SEPRESTO |
| | 75 WS, , CONFIDOR SC350 |
| Dinotefuran | ZYLAM ®, TRANSTECT, SAFARI ®, VENOM ® |
| Clothianidin | ARENA ®, CLUTCH ® WDG, PONCHO ®, |
| | SEPRESTO 75 WS |
| Thiacloprid | CALYPSO, BISCAYA ® |
| Thiamethoxam | ACTARA ®, CRUISER ®, MAXIDE, MERIDIAN ®, |
| | OPTIGARD ®, DURIVO ® |
| Acetamiprid | ASSAIL ®, RESCATE ®, TRISTAR ® |

Pollinators' exposure to neonicotinoids can occur through multiple pathways including ingestion, contact (e.g., contaminated nesting material, plants, soil, water), and inhalation (aerosols). At low concentrations of neonicotinoids, sublethal effects can occur. Sublethal effects involve modifications of honeybee behavior and physiology (e.g., immune system). They do not directly cause the death of the individual or the collapse of the colony but may become lethal in time and/or may make the colony more sensitive (e.g., more prone to diseases), which may contribute to its collapse. For instance, an individual with memory, orientation or physiological impairments might fail to return to its hive, dying from hunger or cold. It has been reported that sub-lethal effects of neonicotinoids exist on neurophysiology, larval development, moulting, adult longevity, immunology, fecundity, sex ratio, mobility, navigation and orientation, feeding behavior, oviposition behavior, and learning.

In some embodiments, there can be a synergism among stressors that negatively impact insect pollinators. A synergistic effect occurs when the effect of a combination of stressors is higher than the sum of the effect of each stressor alone. When neonicotinoids are combined with certain fungicides (e.g., azoles, such as prochloraz, or anilides, such as metalaxyl) or other agrochemicals that block cytochrome p450 detoxification enzymes, their toxicity increases by a factor from 1.52 to 1141 depending on the combination. Synergy has also been demonstrated for neonicotinoids and infectious agents. Prolonged exposure to a non-lethal dose of neonicotinoids renders beehives more susceptible to parasites such as Nosema ceranae. Therefore, in some embodiments, the disclosed compositions can be useful to protect against synergistic effects of pesticides and other pollinator stressors (e.g., parasites).

b. Carbamates and Organophosphates

Impairment of a cognitive function can be induced by exposure to a carbamate or organophosphate. Thus, the disclosed compositions may also be useful to protect against harmful cognitive effects of one or more carbamates or organophosphates. In some embodiments, the pesticide is a carbamate or organophosphate.

Organophosphates are phosphoric acid esters or thiophosphoric acid esters. When developed in the 1930s and 1940s, their original compounds were highly toxic to mammals. Organophosphates manufactured since then are less toxic to mammals but toxic to target organisms, such as insects. Malathion, dibrom, chlorpyrifos, temephos, diazinon and terbufos are exemplary organophosphates.

Carbamates are esters of N-methyl carbamic acid. Aldicarb, carbaryl, propoxur, oxamyl and terbucarb are exemplary carbamates. Carbamates are structurally and mechanistically similar to organophosphate insecticides but differ in action from the organophosphates in that the inhibitory effect on cholinesterase is generally brief.

Although carbamate and organophosphate pesticides differ chemically, they act similarly. When applied to crops or directly to the soil as systemic insecticides, organophosphates and carbamates generally persist from only a few hours to several months. The organophosphates and carbamates are potent inhibitors of acetylcholinesterase, thereby inhibiting the normal breakdown of Acetylcholine (ACh). ACh is a neurotransmitter, a chemical produced by a neuron that transmits signals from that neuron to another neuron, an exocrine gland, or a muscle. Importantly, whereas ACh prevails as a neurotransmitter at the neuromuscular junction in vertebrates with less prevalence in the central nervous system, it is the main neurotransmitter in the nervous system of invertebrates. ACh is released in the junction (synapse) between two cells (a neuron and a muscle or two neurons) where it binds to its receptor on the target cell (the postsynaptic terminal), inducing its activation and relaying the signal.

Acetylcholinesterase (AChE) is an enzyme located in the intercellular space that is responsible for ACh degradation. Organophosphates and carbamates block the site where the neurotransmitter attaches to the Acetylcholinesterase leading to the buildup of ACh and continuous stimulation of the receptors on the target cells. In vertebrates, this results in parasympathetic and sympathetic overstimulation and eventual muscle paralysis. Overstimulation also underlies effects in invertebrates.

c. Fipronil

In some embodiments the pesticide is fipronil. Fipronil targets two main receptors in the insect brain. Fipronil can bind to GABA receptors or to Glutamate ionotropic receptors (chloride channels). GABA is a neurotransmitter that activates chloride channels, thus inducing a hyperpolarization (i.e. making more negative the neuron membrane) and depressing the neural activity. On the other hand, a population of glutamate ionotropic receptors in the insect brain can allow the movement of chloride, also leading to hyperpolarization. Thus, these networks act as modulatory of the activity during information processing. As fipronil targets GABAergic and glutamatergic receptors it acts, unlike other pesticides, on inhibitory, not excitatory, networks. Fipronil may affect the dopaminergic network and thus motor control and the value assigned to a reward. Fipronil effects include mitochondrial impairment, disrupting processing of information.

d. Sulfoximine Derivatives

In some embodiments the pesticides is a derivative of sulfloxamines, such as Sulfoxaflor. Similar to neonicotinoids, Sulfoxaflor targets nicotinic cholinergic receptors (nAChR) being a competitor agonist of ACh. Major impairments at sublethal levels include motor control and reproduction.

C. Controls

The methods disclosed herein typically include comparing the level of a biological parameter in the subject pollinator to a control. Suitable controls will be known to one of skill in the art. Controls can include, for example, standards obtained from subjects not exposed to compositions containing one or more phenolic compounds. Controls can also include the level of a biological parameter at a specific reference point, for example, before administration of compositions containing one or more phenolic compounds. A control can be a single or pooled or averaged values assessed/assayed equivalently to the experimental subject. Reference indices can be established by using pollinators that have been exposed to one or more pesticides and that exhibit one or more cognitive impairments with different known severities or prognoses.

D. Methods of Making

Methods for the production of the disclosed compositions including food, feed additive or supplements, and nutraceuticals are provided. Exemplary feed include feed for honeybees (e.g., sugar syrup, patties). The methods can include the steps of incorporating one or more of the phenolic compounds into the feed product or feed supplement product during the preparation of the feed or supplement. An animal feed or feed supplement for use in the methods described herein may include one or more phenolic compounds, proteins, lipids, carbohydrates, minerals, water, other nutrients or ingredients, or combinations thereof.

The one or more phenolic compounds may be incorporated into the feed product at any stage during the production process including before one or more heating steps or mixing steps.

The following processes may be used alone or in combination, as needed to provide the disclosed compositions and formulations: stirring, mixing, size reduction, and heating. Dry ingredients can be mixed and blended in a high speed mixer/blender to achieve complete mixing and size reduction of the particles. The mixing is carried out sufficient to render the components into a well-dispersed form that is available in a substantially homogeneous manner. If desired, size reduction is carried out sufficient to render the components to be of a size and form so as to remain suspended in the final formulation and be of size acceptable to the mouthparts of an insect, e.g., a bee. In cases where the source ingredients are not greater than about 35 microns, size reduction may not be required. Heating can serve to increase the digestibility and absorption potential for components such as proteins and to destroy microbes, especially those in vegetative phases of their life cycle. Preferably heating is carried out sufficient to accomplish the foregoing but insufficient to cause excessive destruction or breakdown of the nutrients. Mixing and heating parameters for a particular set of circumstances can be readily determined by routine experimentation. In some embodiments, dry and/or solid formulations are mixed with water or liquid formulations to provide the final feed product.

The disclosed compositions and methods of use thereof can be further understood through the following numbered paragraphs.

1. A composition suitable for administration to or ingestion by an insect pollinator comprising one or more phenolic compounds in an effective amount to protect against impairment of a cognitive function of the insect pollinator.

2. The composition of paragraph 1 further comprising a source of carbohydrates, proteins, lipids, vitamins, minerals, water or combinations thereof.

3. The composition of paragraph 2, wherein the source is natural or artificial nectar, honey, sugar, sugar syrup, pollen or pollen substitute, soy flour, soy meal, gluten, skim milk, yeast, pollard, oil, or combinations thereof.

4. The composition of paragraph 2, wherein the source is selected from the group comprising AP23®, BEE-PRO®, FEEDBEE, MEGABEE and ULTRA BEE.

5. The composition of any one of paragraphs 1-4, wherein the one or more phenolic compounds is one or more flavonoids, p-coumaric acid, or a combination thereof.

6. The composition of paragraph 5, wherein the flavonoid is selected from the group comprising flavones, flavanones, flavonols, isoflavones, anthocyanins, flavanols (catechins), chalcones, and neoflavonoids.

7. The composition of paragraph 6, wherein the flavonol is selected from quercetin, rutin, myricetin, kaempferol, fisetin, morin, isorhamnetin, and derivatives or variants thereof.

8. The composition of any one of paragraphs 1-7, wherein the one or more phenolic compounds is selected from the compounds of Table 1 (e.g., isoquercetin, rutin, quercetin, quercitrin, hyperoside, quercimeritrin, baimaside, querciturone, 3-O-methylquercetin, quercetin 3-sambubioside, miquelianin, spiraeoside, isorhamnetin, quercetin 3-O-sulfate, quercetin 7-O-glucoside, quercetin 3-O-Rhamnoside, quercetin 3,4′-diglucoside, and quercetin 3-sophorotrioside) and combinations thereof.

9. The composition of paragraph 7, wherein the flavonol is quercetin, rutin, or a combination thereof.

10. The composition of any one of paragraphs 1-9 for use as a food, feed additive or supplement, or nutraceutical.

11. The composition of any one of paragraphs 1-10, wherein the insect pollinator is a butterfly, moth, fly, beetle, wasp or bee.

12. The composition of paragraph 11, wherein the bee is selected from honeybee, bumblebee, carpenter bee, leafcutter bee, blueberry bee, squash bee, mason bee, orchid bee, stingless bee or sweat bee.

13. The composition of any one of paragraphs 1-12, wherein the cognitive function is selected from learning, memory, attention, decision accuracy, decision speed, navigation, motor activity, sucrose sensitivity, and combinations thereof.

14. The composition of any one of paragraphs 1-13, wherein the impairment is induced by exposure to a pesticide.

15. The composition of paragraph 14, wherein the pesticide is neurotoxic.

16. The composition of paragraph 14 or 15, wherein the pesticide adversely affects GABAergic or glutamatergic neurotransmission, the mushroom bodies, the antennal lobes, the optic lobes or mitochondrial function; increases apoptosis; increases oxidative stress; or combinations thereof in the pollinator.

17. The composition of any one of paragraphs 14-16, wherein the pesticide targets the nicotinic acetylcholine receptor (nAChR).

18. The composition of any one of paragraphs 14-17, wherein the pesticide is a neonicotinoid selected from the group consisting of imidacloprid, thiacloprid, clothianidin, thiamethoxam, acetamiprid, nitenpyram, dinotefuran, and nithiazine.

19. A method of protecting the cognitive function of an insect pollinator, the method comprising administering to an insect pollinator in need thereof a composition of any one of paragraphs 1-18.

20. A method of protecting the learning and/or memory capabilities of an insect pollinator, comprising administering to the insect pollinator a composition of any one of paragraphs 1-18.

21. A method for preventing or reducing impairment of cognitive function in an insect pollinator, comprising administering to the insect pollinator a composition comprising an effective amount of one or more phenolic compounds to prevent or reduce impairment of a cognitive function in the insect pollinator.

22. A method of controlling insect pests comprising treating an area occupied by insect pests and insect pollinators with a pesticide, and administering to the insect pollinator a composition comprising an effective amount of one or more phenolic compounds to prevent or reduce impairment of a cognitive function and/or increase a cognitive function in the insect pollinator.

23. The method of paragraph 22, wherein the area is treated with the pesticide before the composition is administered to the insect pollinators.

24. The method of paragraphs 22, wherein the area is treated with the pesticide after the composition is administered to the insect pollinators.

25. The method of any one of paragraphs 22-24, wherein the area is treated with an effective amount of pesticide to reduce the number of insect pests in the area.

26. The method of any one of paragraphs 22-25, wherein the pesticide is lethal to insect pests.

27. The method of any one of paragraphs 22-26, wherein the area is treated with an amount of pesticide that is sublethal to insect pollinators.

28. The method of any one of paragraphs 20-25, wherein the insect pests are selected from the group consisting of aphids, mites, thrips, whiteflies, leafhoppers, mealybugs, spittlebugs, fleas, termites, scales, and beetles.

29. The method of any one of paragraphs 20-27, wherein the area is treated by pesticide by foliar application, soil injection, tree injection, ground application as a granular or liquid formulation, or as a pesticide-coated seed treatment.

30. The method of any one of paragraphs 21-29, wherein the cognitive function is selected from learning, memory, attention, decision accuracy, decision speed, navigation, motor activity, sucrose sensitivity, and combinations thereof.

31. The method of any one of paragraphs 21-30, wherein the amount of the composition administered is effective to prevent or reduce loss of or reduction in memory, learning, attention, decision accuracy, decision speed, navigation skills, motor activity, or a combination thereof in the insect pollinator upon exposure to a pesticide.

32. The method of any one of paragraphs 21-30, wherein the amount administered is effective to prevent or reduce loss of or reduction in memory, learning, navigation skills, motor activity, or combinations thereof in the insect pollinator compared to an insect pollinator not administered the composition.

33. The method of any one of paragraphs 21-32, wherein the amount administered is effective to prevent or reduce mitochondrial dysfunction, apoptosis and/or oxidative stress in the brain.

34. The method of paragraph 33, wherein the amount administered is effective to prevent or reduce mitochondrial dysfunction, apoptosis and/or oxidative stress in the mushroom bodies.

35. The method of any one of paragraphs 21-34, wherein the one or more phenolic compounds is one or more flavonoids, p-coumaric acid, or a combination thereof.

36. The method of paragraph 35, wherein the flavonoid is selected from the group comprising flavones, flavanones, flavonols, isoflavones, anthocyanins, flavanols (catechins), chalcones, and neoflavonoids.

37. The method of paragraph 36, wherein the flavonol is selected from quercetin, rutin, myricetin, kaempferol, fisetin, morin, isorhamnetin, and derivatives or variants thereof.

38. The method of any one of paragraphs 21-37, wherein the impairment is induced by exposure to a pesticide.

39. The method of paragraph 38, wherein the pesticide is at a sublethal dose.

40. The method of any one of paragraphs 21-22 and 24-39, wherein exposure to the pesticide occurs after administration of the composition.

41. The method of any one of paragraphs 38-40, wherein the pesticide is neurotoxic.

42. The method of any one of paragraphs 38-41, wherein the pesticide adversely affects GABAergic or glutamatergic neurotransmission or the function of mushroom bodies; increases mitochondrial dysfunction, apoptosis or oxidative stress in the brain; or combinations thereof.

43. The method of any one of paragraphs 38-42, wherein the pesticide targets the nicotinic acetylcholine receptor (nAChR).

44. The method of any one of paragraphs 38-43, wherein the pesticide is a neonicotinoid selected from the group consisting of imidacloprid, thiacloprid, clothianidin, thiamethoxam, acetamiprid, nitenpyram, dinotefuran, and nithiazine.

45. The method of any one of paragraphs 38-41, wherein the pesticide is fipronil, carbamate, an organophophate, a derivative of sulfloxamine, or a pyrethroid, optionally wherein the pyrethroid is deltamethrin.

46. The method of any one of paragraphs 21-45, wherein the insect pollinator is a butterfly, moth, fly, beetle, wasp or bee.

47. The method of paragraph 46, wherein the bee is selected from honeybee, bumblebee, carpenter bee, leafcutter bee, blueberry bee, squash bee, mason bee, stingless bee, orchid bees or sweat bee.

48. The method of any one of paragraphs 21-47, wherein the composition is a food, feed additive or supplement, or nutraceutical.

49. The method of paragraph 48, wherein the composition is administered via ingestion.

50. The method of any one of paragraphs 21-49, wherein the one or more phenolic compounds is selected from the compounds of Table 1 (e.g., isoquercetin, rutin, quercetin, quercitrin, hyperoside, quercimeritrin, baimaside, querciturone, 3-O-methylquercetin, quercetin 3-sambubioside, miquelianin, spiraeoside, isorhamnetin, quercetin 3′-O-sulfate, quercetin 7-O-glucoside, quercetin 3-O-Rhamoside, quercetin 3,4′-diglucoside, and quercetin 3-sophorotrioside) and combinations thereof.

51. A method of protecting insect pollinators from a pesticide, the method comprising administering to insect pollinators a composition comprising an effective amount of one or more phenolic compounds to prevent or reduce impairment of a cognitive function and/or increase a cognitive function in the insect pollinator following exposure to a pesticide.

52. The method of paragraph 51, wherein the one or more phenolic compounds is one or more flavonoids, p-coumaric acid, or a combination thereof.

53. The method of paragraph 52, wherein the flavonoid is selected from the group comprising flavones, flavanones, flavonols, isoflavones, anthocyanins, flavanols (catechins), chalcones, and neoflavonoids.

54. The method of paragraph 53, wherein the flavonoid is a favonol.

55. The method of any one of paragraphs 51-54, wherein the one or more phenolic compounds is selected from quercetin, rutin, myricetin, kaempferol, fisetin, morin, isorhamnetin, p-coumaric acid, and derivatives or variants thereof, and combinations thereof.

56. The method of any one of paragraphs 51-54, wherein the one or more phenolic compounds is selected from the compounds of Table 1 (e.g., isoquercetin, rutin, quercetin, quercitrin, hyperoside, quercimeritrin, baimaside, querciturone, 3-O-methylquercetin, quercetin 3-sambubioside, miquelianin, spiraeoside, isorhamnetin, quercetin 3′-O-sulfate, quercetin 7-O-glucoside, quercetin 3-O-Rhamnoside, quercetin 3,4′-diglucoside, and quercetin 3-sophorotrioside) and combinations thereof.

57. The method of any one of paragraphs 51-55, wherein an area occupied by the insect pollinators has been or will be treated with a pesticide.

58. The method of paragraph 57, wherein the area occupied by the insect pollinators is also occupied with insect pests.

59. The method of any one of paragraphs 51-58, further comprising treating the area occupied by the insect pollinators with the pesticide.

60. The method of any one of paragraphs 57-59, wherein the area occupied by the insect pollinators is treated with the pesticide before the composition is administered to the insect pollinators.

61. The method of any one of paragraphs 57-59, wherein the area occupied by the insect pollinators is treated with the pesticide after the composition is administered to the insect pollinators.

62. The method of any one of paragraphs 57-61, wherein the insect pollinators are administered the composition in the same area treated with the pesticide.

63. The method of any one of paragraphs 57-61, wherein the insect pollinators are administered the composition in an area separate from the pesticide.

64. The method of any one of paragraphs 57-63, wherein the area is treated with an effective amount of pesticide to reduce the number of insect pests in the area.

65. The method of any one of paragraphs 57-64, wherein the pesticide is lethal to insect pests.

66. The method of any one of paragraphs 57-65, wherein the area is treated with an amount of pesticide that is sublethal to insect pollinators.

67. The method of any one of paragraphs 51-66, wherein the insect pests are selected from the group consisting of aphids, mites, thrips, whiteflies, leafhoppers, mealybugs, spittlebugs, fleas, termites, scales, beetles, and combinations thereof.

68. The method of any one of paragraphs 51-67, wherein the insect pollinators are selected from butterflies, moths, flies, beetles, wasps, bees, and combination thereof.

69. The method of paragraph 68, wherein the bees are selected from honeybees, bumblebees, carpenter bees, leafcutter bees, blueberry bees, squash bees, mason bees, stingless bees, orchid bees, sweat bees, and combinations thereof.

70. The method of any one of paragraphs 51-69, wherein the cognitive function is selected from learning, memory, attention, decision accuracy, decision speed, navigation, motor activity, sucrose sensitivity, and combinations thereof.

71. The method of any one of paragraphs 51-70, wherein the amount of the composition administered is effective to prevent or reduce loss of or reduction in memory, learning, attention, decision accuracy, decision speed, navigation skills, motor activity, or a combination thereof in the insect pollinator upon exposure to a pesticide.

72. The method of any one of paragraphs 51-71, wherein the amount administered is effective to prevent or reduce loss of or reduction in memory, learning, navigation skills, motor activity, or combinations thereof in the insect pollinator compared to an insect pollinator not administered the composition.

73. The method of any one of paragraphs 51-72, wherein the amount administered is effective to prevent or reduce mitochondrial dysfunction, apoptosis and/or oxidative stress in the brain.

74. The method of any one of paragraphs 51-73, wherein the amount administered is effective to prevent or reduce mitochondrial dysfunction, apoptosis and/or oxidative stress in the mushroom bodies.

75. The method any one of paragraphs 51-74, wherein the impairment is induced by exposure to the pesticide.

76. The method any one of paragraphs 51-75, wherein the pesticide is at a sublethal dose.

77. The method of any one of paragraphs 51-76, wherein the pesticide is neurotoxic to insect pollinators.

78. The method of any one of paragraphs 51-77, wherein the pesticide adversely affects GABAergic or glutamatergic neurotransmission or the function of mushroom bodies; increases mitochondrial dysfunction, apoptosis or oxidative stress in the brain; or combinations thereof.

79. The method of any one of paragraphs 51-77, wherein the pesticide targets the nicotinic acetylcholine receptor (nAChR).

80. The method of any one of paragraphs 51-79, wherein the pesticide is a neonicotinoid.

81. The method of any one of paragraphs 51-80, wherein the pesticide is imidacloprid, thiacloprid, clothianidin, thiamethoxam, acetamiprid, nitenpyram, dinotefuran, nithiazine, fipronil, carbamate, an organophophate, a derivative of sulfloxamine, or a pyrethroid, optionally wherein the pyrethroid is deltamethrin.

82. The method of any one of paragraphs 51-81, wherein exposure to the pesticide occurs after administration of the composition.

83. The method of any one of paragraphs 51-82, wherein the composition is a food, feed additive or supplement, or nutraceutical.

84. The method of any one of paragraphs 51-83, wherein the composition is administered via ingestion.

85. The method of any one of paragraphs 51-84, wherein the pesticide is delivered by foliar application, soil injection, tree injection, ground application as a granular or liquid formulation, or as a pesticide-coated seed treatment.

86. The method of any one of paragraphs 51-85, wherein the one or more phenolic compounds is quercetin, rutin, kaempferol, p-coumaric acid, or a combination thereof.

87. A composition suitable for administration to or ingestion by an insect pollinator comprising one or more phenolic compounds in an effective amount to protect against impairment of a cognitive function of the insect pollinator.

88. The composition of paragraph 87 further comprising a source of carbohydrates, proteins, lipids, vitamins, minerals, water or combinations thereof.

89. The composition of paragraph 88, wherein the source is natural or artificial nectar, honey, sugar, sugar syrup, pollen or pollen substitute, soy flour, soy meal, gluten, skim milk, yeast, pollard, oil, or combinations thereof.

90. The composition of paragraphs 88 or 89, wherein the source is selected from the group comprising AP23®, BEE-PRO®, FEEDBEE, MEGABEE and ULTRA BEE.

91. The composition of any one of paragraphs 87-90, wherein the one or more phenolic compounds is one or more flavonoids, p-coumaric acid, or combination thereof.

92. The composition of paragraph 91, wherein the flavonoid is selected from the group comprising flavones, flavanones, flavonols, isoflavones, anthocyanins, flavanols (catechins), chalcones, and neoflavonoids.

93. The composition of paragraph 92, wherein the flavonol is selected from quercetin, rutin, myricetin, kaempferol, fisetin, morin, isorhamnetin, and derivatives or variants thereof.

94. The composition of any one of paragraphs 87-93, wherein the one or more phenolic compounds is selected from the compounds of Table 1 (e.g., isoquercetin, rutin, quercetin, quercitrin, hyperoside, quercimeritrin, baimaside, querciturone, 3-O-methylquercetin, quercetin 3-sambubioside, miquelianin, spiraeoside, isorhamnetin, quercetin 3-O-sulfate, quercetin 7-O-glucoside, quercetin 3-O-Rhamnoside, quercetin 3,4′-diglucoside, and quercetin 3-sophorotrioside) and combinations thereof.

95. The composition of any one of paragraphs 87-94, wherein the one or more phenolic compounds is selected from quercetin, rutin, myricetin, kaempferol, fisetin, morin, isorhamnetin, p-coumaric acid, and derivatives or variants thereof, and combinations thereof.

96. The composition of any one of paragraphs 87-95 for use as a food, feed additive or supplement, or nutraceutical.

97. The composition of any one of paragraphs 87-96, wherein the insect pollinator is a butterfly, moth, fly, beetle, wasp or bee.

98. The composition of any one of paragraphs 87-97, wherein the bee is selected from honeybee, bumblebee, carpenter bee, leafcutter bee, blueberry bee, squash bee, mason bee, orchid bee, stingless bee, or sweat bee.

99. The composition of any one of paragraphs 87-98, wherein the cognitive function is selected from learning, memory, attention, decision accuracy, decision speed, navigation, motor activity, sucrose sensitivity, and combinations thereof.

100. The composition of any one of paragraphs 87-99, wherein the impairment is induced by exposure to a pesticide.

101. The composition of any one of paragraphs 87-100, wherein the pesticide is neurotoxic.

102. The composition of any one of paragraphs 87-101, wherein the pesticide adversely affects GABAergic or glutamatergic neurotransmission, the mushroom bodies, the antennal lobes, the optic lobes or mitochondrial function; increases apoptosis; increases oxidative stress; or combinations thereof in the pollinator.

103. The composition of any one of paragraphs 87-102, wherein the pesticide targets the nicotinic acetylcholine receptor (nAChR).

104. The composition of any one of paragraphs 87-103, wherein the pesticide is a neonicotinoid.

105. The composition of any one of paragraphs 87-104, wherein the pesticide is imidacloprid, thiacloprid, clothianidin, thiamethoxam, acetamiprid, nitenpyram, dinotefuran, nithiazine, fipronil, carbamate, an organophophate, a derivative of sulfloxamine, or a pyrethroid, optionally wherein the pyrethroid is deltamethrin.

106. A method of protecting the cognitive function of an insect pollinator, the method comprising administering to an insect pollinator in need thereof a composition of any one of paragraphs 87-105.

107. A method of protecting the learning and/or memory capabilities of an insect pollinator, comprising administering to the insect pollinator a composition of any one of paragraphs 87-106.

108. A method for preventing or reducing impairment of cognitive function in an insect pollinator, comprising administering to the insect pollinator a composition comprising an effective amount of one or more phenolic compounds to prevent or reduce impairment of a cognitive function in the insect pollinator.

109. The composition or method of any one of paragraphs 1-108, wherein the one or more phenolic compounds is quercetin, rutin, kaempferol p-coumaric acid, or a combination thereof.

The present invention will be further understood by reference to the following non-limiting examples.

### EXAMPLES

### Example 1: Flavonoids Enhance Learning and Memory and Protect Honeybees Against Neonicotinoid-Induced Impairment

### Materials and Methods

Honeybee Collection and Maintenance

Honeybees (Apis mellifera scutellata) were captured from established hives at the Apiary of the Pontificia Universidad Javeriana (Bogota, Colombia). Foragers were collected while leaving to forage between 8:00 am and 10:00 am (Matsumoto et al., J Neurosci Methods. 211(1):159-67. 2012). Bees were maintained in individual plastic chambers under laboratory conditions (photoperiod followed seasonal pattern; relative humidity: 63%; temperature: 18° C.). Food was provided once a day in a volume of 20 μL and varied in composition according to experimental treatments.

Honeybee Experimental Treatments

Bees were randomly allocated to one of the following treatments:

Control (2M sugar water): fed once a day (20 μL) for three consecutive days;

Quercetin (6.6 mM quercetin (Sigma-Aldrich: Q4951)-sucrose-water with DMSO 1% (Sigma-Aldrich: D8418): fed once a day for three days;

Rutin (1 mM rutin (Sigma-Aldrich R5143)-sucrose-water with DMSO 1%: fed once a day for three days;

Imidacloprid (1.16 nM Imidacloprid (Bayer)-sucrose-water: fed as a single dose (20 μL) 1.5 h before training;

Quercetin-Imidacloprid: bees received the quercetin treatment (see above) and were fed Imidacloprid 1.5 h before training; and

Rutin-Imidacloprid: bees received the rutin treatment (see above) and were fed Imidacloprid 1.5 h before training.

Training

Bees were trained using classical conditioning of the proboscis extension response (PER), a broadly used method to study learning and memory in bees. Briefly, bees received a soft current of clean air for 15 seconds to allow bees to accommodate. Then a soft current of 1-hexanol (Sigma-Aldrich: 471402) was injected for 10 seconds. Six seconds after injection of 1-hexanol, one of the antennae was stimulated with sucrose water (50% w/w), which leads to the reflexive extension of the proboscis, and the bee was allowed to drink for 4 seconds. Thus, the reward and the stimulus overlapped for four seconds. This sequence, recognized as a training trial, was repeated eight times. Time was determined using a metronome (2 Hz). Memory retention was tested 24 hours after the eight training trials. During each trial, whether the bee extended the proboscis to the sole presentation of 1-hexanol and the time to exhibit the response were recorded. 12 bees were trained each session. Training was conducted by an experimenter blind to treatment.

Data Collection and Statistical Analysis

Learning was assessed from the total number of conditioned responses across the eight trials. Thus, each bee received a learning score between 0-7 (by definition, bees cannot show a learned response at the first trial). After 24 h, memory retention was assessed as the response to the presentation of 1-hexanol without a sucrose reward. Latency of response was quantified as the time between the onset of 1-hexanol stimulation and the onset of a response that led to a full extension of proboscis. Only bees being responsive to sucrose water across all training trials and the memory retention test were considered for analyses. Learning scores were compared across treatments using a Mann-Whitney test as data were not normally distributed (Shapiro-Wilk W test for the hypothesis that the data is from a Normal distribution: P<0.05 for all treatments). Comparisons of the learning score of bees across all treatments relative to Control bees were conducted using the Steel method adjusted as one or two-tailed tests. Additional post hoc comparisons were performed between Imid and Quer+Imid bees and between Imid and Rut+Imid bees. Memory retention was compared across groups using an overall Chi-Square test and independent tests for planned comparisons were performed using adjusted Wald tests for proportions. In all cases error due to multiple comparisons was controlled using the False Discovery Rate one stage method and adjusted p-values (q-values) are presented (Benjamini & Hochberg, Journal of the Royal Statistical Society. Series B (Methodological), 57(1):289-300. 1995; Pike, N., Methods in Ecology and Evolution, 2(3):278-282. 2011). All analyses were conducted using JMP v.14.0 (SAS Institute Inc).

### Results

The protective effect of two flavonoids, Quercetin and Rutin, against the negative effects of the neonicotinoid, Imidacloprid was investigated in the honeybee.

The acquisition performance and memory retention of honeybees exposed to Imidacloprid, and of honeybees prophylactically treated with Quercetin and its derivative, Rutin, was evaluated. During the first day, bees were randomly assigned to one of three treatments: bees fed with a single daily dose of sugar-water and bees fed with a single daily dose of either Quercetin or Rutin. On the fourth day of this schedule, bees within each group were randomly assigned to one of two treatments 1.5 hours before training: bees fed with sugar-water and bees fed with a solution of Imidacloprid. Thus, six treatment conditions were compared: Control (only receiving sucrose-water), Quercetin (Quer), Rutin (Rut), Quercetin+imidacloprid (Quer+imid), Rutin+imidacloprid (Rut+imid) and Imidacloprid (Imid).

For learning and retention, bees were evaluated using classical conditioning of the proboscis extension response (PER), a widely used method for study of honeybee bee cognition (Takeda, J. Insect. Physiol., 6:168-179. 1961; Bitterman, J. Comp. Psychol., 97:107-119. 1983). Briefly, an experimenter blind to treatment assignments, trained bees to eight paired presentations of a stimulus (1-hexanol) and a reward (sugar water). Over presentations, bees learning the association exhibited a PER after the sole presentation of the trained stimulus (a ‘conditioned’ PER). After 24 hours, bees were tested in their response to the presentation of the sole stimulus, thus evaluating retention of the learnt association. Hence, bees with the best performances should exhibit more conditioned PERs during training and to exhibit a conditioned PER response after 24 hours.

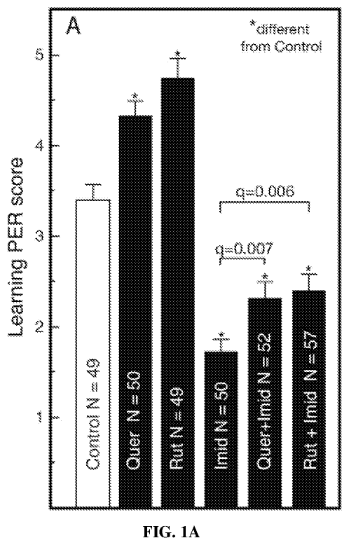

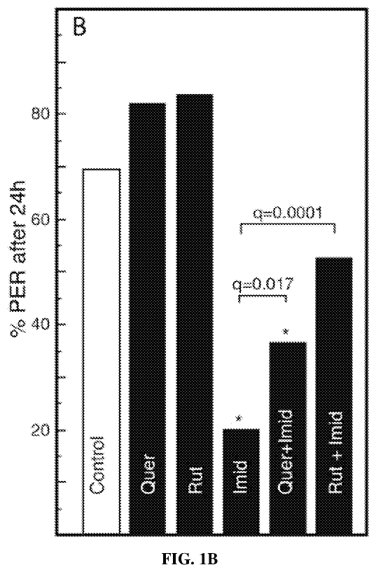

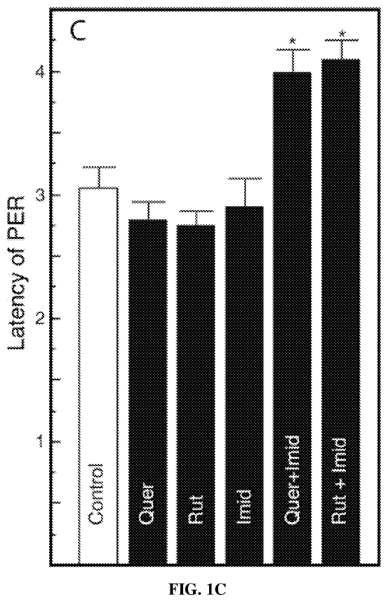

A significant effect of treatment on learning acquisition (Kruskal-Wallis test: X₅=114.74, p<0.0001; FIG. 1A) and memory retention (Likelihood ratio: X₅=71.26, p<0.0001; FIG. 1B) was found. Bees receiving quercetin and rutin exhibited significantly higher learning scores (Rut: Z=4.23, q=0.0002; Quer: Z=3.52, q=0.003) but not memory retention scores than Control bees (Adjusted Wald test: Rut: Proportion difference=−0.14, q=0.128; Quer: Proportion difference=−0.13, q=0.1527).

In contrast, bees receiving only imidacloprid exhibited learning scores significantly lower than Control bees (Imid: Z=−5.91, q=0.0002). Moreover, after 24 h, the probability of exhibiting a conditioned PER was significantly lower relative to Control bees (Adjusted Wald test: Proportion difference=0.49, q=0.0002). Only 20% of the bees exposed to imidacloprid exhibited a conditioned PER; whereas 69.4% of the Control bees responded during the retention test. The odds of remembering were estimated to be 9.1 times higher (95% CI=3.6-22.8) for Control bees relative to Imid bees.

Most importantly, bees prophylactically fed with rutin or quercetin had significantly higher learning scores than bees in the Imid group (Rut: Z=2.73, q=0.006; Quer: Z=2.45, q=0.007; FIG. 1A). Moreover, after 24 h, bees in the Rut+Imid (Adjusted Wald test: Proportion difference=−0.33, q=0.0001) and the Quer+Imid group (Adjusted Wald test: Proportion difference=−0.16, q=0.017) had a higher probability of exhibiting a conditioned PER relative to Imid bees (FIG. 1B). 52.6% in the Rut+Imid group and 36.5% in the Quer+Imid group exhibited a conditioned response after 24 h. The odds of remembering were estimated to be 4.4 times higher (95% CI=1.9-10.6) for Rut+Imid bees and 2.3 times higher (95% CI=0.9-5.6) for Quer+Imid relative to Imid bees. However, bees in the Rut+Imid and Quer+Imid groups exhibited significantly lower learning scores than Control bees (Rut+Imid: Z=−3.38, q=0.003; Quer+Imid: Z=−3.67, q=0.003; FIG. 1A). After 24 h, bees in Quer+Imid but not in the Rut+Imid had a significantly lower probability of exhibiting a conditioned PER than Control bees.

A significant effect of treatment on latency of response during learning acquisition (Kruskal-Wallis test: X₅=54.72, p<0.0001; FIG. 1C) was also found. Bees in the Rut+Imid and Quer+Imid groups exhibited significantly longer latencies of response relative to Control bees (Rut+Imid: Z=4.07, q=0.001; Quer+Imid: Z=3.60, q=0.004; FIG. 1C). Latency of response across all other treatments was not significantly different from Control (Rut: Z=−0.91, q=0.847; Quer: Z=−0.95, q=0.847; Imid: Z=−0.88, q=0.847; FIG. 1C).

Together, these results show that the flavonoids rutin and quercetin protect honey bees against the cognitive impairment in learning and memory produced by imidacloprid (thought to produce short term effects) and its metabolites (thought to produce long-term effects).

### Example 2: Flavonoid Protects Bumblebee Learning and Memory Against Neonicotinoid-Induced Impairment

Materials and Methods

Bumblebee Collection, Maintenance and Experimental Treatments

One colony of the bumblebee Bombus impatiens (Koppert Inc) was kept in laboratory conditions. Bees received pollen inside the nests and foraged to a 1M sugar-water feeder outside of the nest. Bees were collected while on the feeder and iced anesthetized to then be yolked restrained in plastic pipettes. Bees were maintained in the pipettes for the entire duration of the experiment (five days).

Bumblebees were randomly allocated to one of the following treatments:

Control: 1M sugar water (20 μL) fed twice a day for three consecutive days;

Rutin: 1 μM Rutin (Sigma-Aldrich R5143) diluted in 1M sugar water (20 μL) fed twice a day for three days before training on the fourth day;

Imid: fed with 1M sugar water (20 μL) twice a day for three days and fed with 20 μL of 5 nM Imidacloprid (Prime Source LLC, IN, USA) in 1M sugar water 2 hours before training on the fourth day; and

Rutin+Imid: Bees fed with 1 μM Rutin diluted in 1M sugar water (20 μL) for three days before training and receiving 20 μL of 5 nM Imidacloprid in 1M sugar water 2 hours before training on the fourth day.

Training

All bees were trained using olfactory conditioning of the proboscis extension response (PER). Briefly, bees were exposed to a presentation of 1-nonanol (Sigma-Aldrich) for 10 s. After 7 s of the onset of the odor presentation, the antenna was stimulated with 1.5 M sugar-water to elicit the PER. Following the PER, the bee was allowed to drink sugar-water for 3 s. Each paired presentation was considered a training trial and each individual received eleven training trials with an average intertrial interval of 10 minutes. After training, all bees were fed 20 μL of 1M sugar water and maintained in plastic boxes for 24 h until the retention test. For each trial whether a conditioned PER was exhibited and its latency in seconds using a metronome (2 Hz) was recorded. Twenty-four hours after the last training trial the bees were presented with the conditioned odor for 10 s. Whether a bee exhibited a conditioned PER was recorded. Bees not exhibiting a conditioned PER were stimulated with sugar-water in order to test motivation.

Data Collection and Statistical Analysis

Performance for each individual was quantified as the number of conditioned PER responses across the trials. Thus, for each bee a score between zero and ten (0-10) was recorded. The Acquisition scores across the four treatments were compared using a Kruskal-Wallis test since the data did not follow a normal distribution and could not be normalized after transformations.

Multiple comparisons were then run using Wilcoxon-tests between groups using single or two-tailed t-tests depending upon predictions (bees on all treatments were predicted to perform better than bees in the Imidacloprid group). Error due to multiple comparisons was controlled using the False Discovery Rate method (FDR-two stage sharpened method). In all cases corrected p-values are presented.

Acquisition learning (i.e. learning curves) was compared between bees fed with imidacloprid vs. protected bees using Repeated measures.

Retention was quantified as a nominal variable (yes/no). Data were compared across treatments using a Chi-square test, followed by multiple comparisons between pairs of treatments. Error due to multiple comparisons was controlled using the FDR as indicated above. In all cases corrected p-values are presented.

Latency was quantified as a continuous variable between 0.5 s and 10 s. Data were compared across treatments using an ANOVA following a Log transformation to normalize the distribution (Shapiro-Wilk W Test after log transformation: W=0.99, p=0.55), followed by multiple comparisons between groups using two-tailed t-test. Error due to multiple comparisons was controlled using the FDR method as indicated above. In all cases corrected p-values are presented.

### Results

The protective effect of the flavonol rutin against the negative sublethal effects of imidacloprid was investigated using the conditioning of the proboscis extension response.

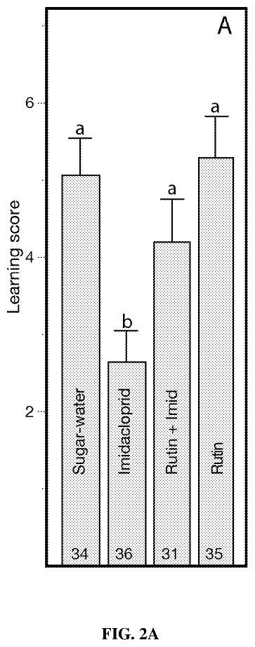

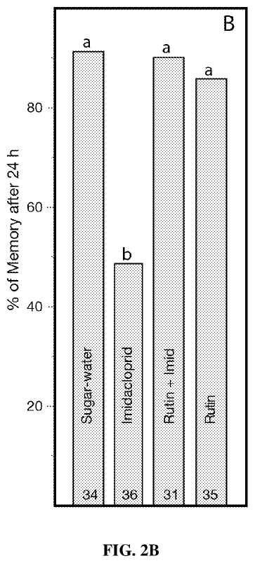

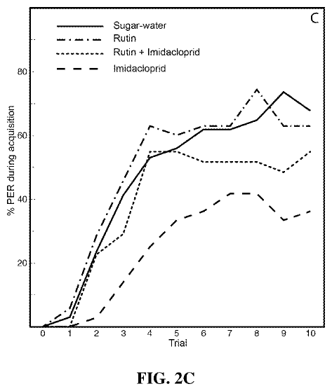

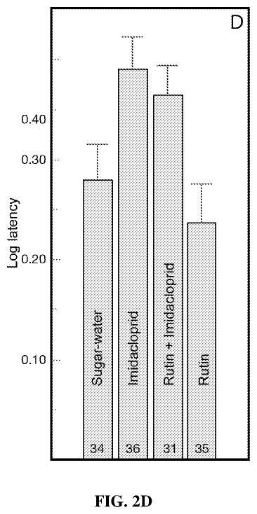

The performance score across treatments did not exhibit a normal distribution (Shapiro-Wilk W test: W: 0.927, p<0.0001) and could not be normalized using transformation. Thus, non-parametric tests were used for comparisons across treatments and for multiple comparisons. Results indicated that exposition to the treatments significantly affected the performance score (Kruskal-Wallis: Chi-square: 15.81 DF: 1, p: 0.0012: FIG. 2A). Bees exposed to imidacloprid had scores significantly lower than Control bees (Wilcoxon-test: Z=−3.37, p=0.0006; FIG. 2A). Most importantly bees prophylactically fed with Rutin and subsequently exposed to 5 nM imidacloprid exhibited performances that were significantly higher than bees only exposed to imidacloprid (Wilcoxon-test: Z=2.0, p=0.024; FIG. 2A) and did not significantly differ from Control bees (Wilcoxon-test: Z=−1.09, p=0.176; FIG. 2A) or bees only fed with rutin (Wilcoxon-test: Z=1.41, p=0.126; FIG. 2A). Moreover, learning across trials was significantly lower in bees fed with imidacloprid (Imidacloprid group) than prophylactically fed with rutin and then exposed to imidacloprid (Rutin+Imidacloprid) (Repeated measures MANOVA: F₁: 5.06, p=0.028; FIG. 2C). Interestingly, the latency of response was also significantly affected by the treatments (Oneway ANOVA: F_{3,105}=3.93, p=0.011) (FIG. 2D). The bees exposed only to imidacloprid exhibited latencies significantly longer than Control bees (p=0.042) and than bees fed with Rutin (p=0.015), but not different from bees prophylactically fed with Rutin (p=0.44).

After 24 h memory retention was also significantly affected by the feeding treatment (Likelihood ratio-test: Chi Square: 21.20, p<0.0001). Bees in the Imidacloprid group were the only group exhibiting a different level of memory retention. Bees in the Imidacloprid group exhibited memory retention significantly lower than bees fed with only sugar water (Control group; p=0.0002, FIG. 2B), bees prophylactically receiving Rutin (Rutin+Imidacloprid group; p=0.0002; FIG. 2B) and bees fed only with Rutin (Rutin group; p=0.0017; FIG. 2B).

Hence, these results show the protective effect against a neonicotinoid on both learning and memory provided by the prophylactic administration of a flavonoid, in this case Rutin.

### Example 3: Flavonoid Protects Drosophila Fly Motor Activity Against Neonicotinoid-Induced Impairment

### Materials and Methods

Experimental Treatments

Standard methods were relied on to test motor activity and motor impairments in Drosophila melanogaster (Jimenez-Del-Rio et al. 2010. Neurochem Res 35:227-238). The method relies on the negative geotropic response (i.e. preference to walk up when standing on a wall) of insects. Four-day-old (post eclosion) D. melanogaster females were randomly assigned to one of four treatments: Control (fed on 5% sugar-water), Rutin 0.1 (fed on a solution 0.1 μM rutin in 5% sugar-water), Rutin 1 (fed on a solution 1 μM rutin in 5% sugar-water), Rutin 10 (fed on a solution 10 uM rutin in 5% sugar-water). After three days flies from each group were randomly assigned to one of two treatments: 5% sugar-water or 0.1 μM imid in 5% sugar-water. Thus, flies were in one of eight treatments: Control, Imid, Rut 0.1, Rut 0.1+Imid, Rut 1, Rut 1+Imid, Rut 10, Rut 10+Imid. Flies were exposed to their specific treatment for 22 h.

Testing Motor Activity

Following standard methods, flies were transferred to a clean vial and a threshold of 5 cm from the bottom was set on the vial. The bottom of the vial was knocked on the table to assure all flies were moved to the bottom. The number of flies that crossed the threshold within 6 seconds were counted. Data were analyzed using a Chi-square with specific predictions (one-tail tests) for planned comparisons. Error associated with multiple comparisons was corrected using the False Discovery Rate-method (two stage sharpen method).

### Results

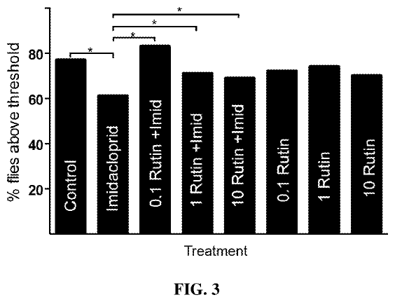

Flies receiving imidacloprid exhibited lower performance than flies in the control group (Chi-square: 7.34, q=0.0058; FIG. 3), demonstrating the neuromotor impairment. Remarkably, all flies prophylactically fed with rutin were protected against this impairment. Flies receiving imidacloprid exhibited a motor performance significantly lower than flies prophylactically receiving 0.1 μM (Chi-Square: 22.58, q<0.0001), 1 NM (Chi-Square: 3.91, q=0.025) or 10 μM (Chi-Square: 4.82, q=0.015) rutin (FIG. 3). The results are strong evidence that a flavonol, in this case rutin, can effectively protect neuromotor function in an insect, in this case a fruit fly.

### Example 4: Ad Libitum Self-Administration of a Flavonoid Protects Bumblebee Learning and Memory Against Acute Administration of a Neonicotinoid and Fipronil

### Materials and Methods

Bumblebee Collection, Maintenance and Experimental Treatments

One colony of the bumblebee Bombus impatiens (Koppert Inc) was kept in laboratory conditions. Bees received pollen inside the nests and foraged to a 1M sugar-water feeder outside of the nest. Bees were collected while on the feeder and iced anesthetized to then be randomly assigned to one of two plastic containers where ad libitum self-administration of one of two solutions was allowed to 10 bees: i) 1 M sucrose water or ii) 1 μM Rutin (Sigma-Aldrich R5143) diluted in 1M sucrose water. On the night of the third day, the feeder was removed to starve the bees. On the fourth day, the bees were iced anesthetized and yolked restrained in plastic pipettes. Bees were maintained in the pipettes for the rest of the experiment (two days). One hour after harnessing, the bees were randomly assigned to one of three treatments: i) 20 μL of 1M sucrose water, ii) 20 μL of 1M sucrose water+5 nM imidacloprid, iii) 20 μL of 1M sucrose water+6 nM fipronil. Thus, overall bees belonged to one of six treatments:

Control: ad libitum sucrose water for three days and 20 μL of sucrose water before training

Rutin: ad libitum 1 μM Rutin for three days and 20 μL of sucrose water before training

Imid: ad libitum sucrose water for three days and 20 μL of 5 nM Imidacloprid in 1M sugar water 2 hours before training

Rutin+Imid: ad libitum 1 μM Rutin for three days and 20 μL of 5 nM Imidacloprid in 1M sugar water 2 hours before training

Fipronil: ad libitum sucrose water for three days and 20 μL of 5 nM fipronil in 1M sucrose water 2 hours before training

Rutin+Fipronil: ad libitum 1p M Rutin for three days and 20 μL of 6 nM fipronil in 1M sugar water 2 hours before training

Training

All bees were blind-trained using olfactory conditioning of the proboscis extension response (PER). Briefly, bees were exposed to a presentation of 1-nonanol (Sigma-Aldrich) for 10 s. After 7 s of the onset of the odor presentation, the antenna was stimulated with 1.5 M sugar-water to elicit the PER. Following the PER, the bee was allowed to drink sugar-water for 3 s. Each paired presentation was considered a training trial and each individual received eleven training trials with an average intertrial interval of 10 minutes. After training, all bees were fed 20 μL of 1M sugar water and maintained in plastic boxes for 24 h until the retention test. For each trial whether a conditioned PER was exhibited and its latency in seconds using a metronome (2 Hz) was recorded. Twenty-four hours after the last training trial the bees were presented with the conditioned odor for 10 s. Whether a bee exhibited a conditioned PER was recorded. Bees not exhibiting a conditioned PER were stimulated with sucrose water in order to test motivation.

Data Collection and Statistical Analysis

Learning for each individual was quantified as the number of conditioned PER responses across the trials. Thus, for each bee a learning score between zero and ten (0-10) was recorded. The scores across the six treatments were compared using a Kruskal-Wallis test since the data did not follow a normal distribution and could not be normalized after transformations.

Multiple comparisons were then run comparing learning score relative to Control bees using a non-parametric one or two-tailed Steel Method depending upon predictions. These comparisons informed us about the predicted impairment produced by the insecticides, the predicted innocuous effect of the flavonoid and the potential level of protection. Error due to multiple comparisons was controlled using the False Discovery Rate method (FDR-two stage sharpened method). In all cases corrected p-values (q-values) are presented.

Memory retention was quantified as a nominal variable (yes/no). Data were compared across treatments using a Chi-square test, followed by multiple comparisons of all treatments vs. Control. Error due to multiple comparisons was controlled using the FDR as indicated above. In all cases corrected p-values (q values) are presented.

Latency was quantified as a continuous variable between 0.5 s and 10 s. Data were compared across treatments using an ANOVA following a Log transformation to normalize the distribution (Shapiro-Wilk W Test after log transformation: p>0.05 for all treatments for the null hypothesis that data is from a normal distribution), followed by multiple comparisons between all treatments and the Control group. Error due to multiple comparisons was controlled using the FDR method as indicated above. In all cases corrected p-values (q-values) are presented.

### Results

The protective effect of the flavonol rutin against the negative sublethal effects of imidacloprid—targeting nAChR- and fipronil—targeting GABA and dopaminergic receptors—was investigated using the conditioning of the proboscis extension response.

The data was analyzed for outliers using Mahalanobis distances including the learning score and memory retention as variables. 30 bees were excluded and final analyses were conducted on 145 bees distributed across the six treatments: Control (N=28), Rut (N=26), Imid (N=23), Rut+Imid (N=21), Fipro (N=26), Rut+Fipro (N=25).

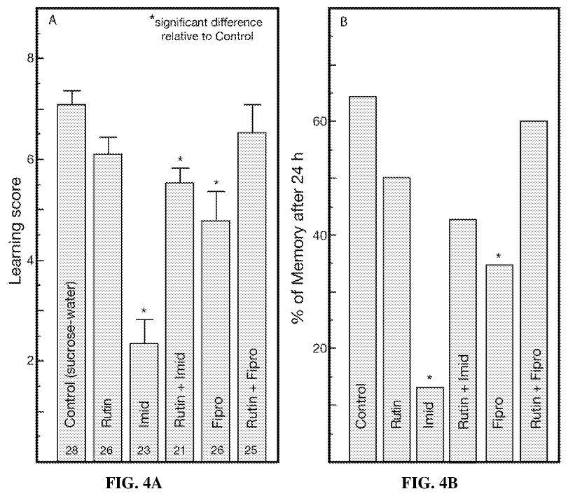

Results show that learning score was significantly affected by treatment (Kruskal-Wallis test: X₅=42.66, p<0.0001). Bees in the Imid (z=−5.411, q=0.0001), Fipro (z=−2,829, q=0.008) and Rut+Imid group (z=−3.208, q=0.008) exhibited a learning score significantly lower than Control bees (FIG. 4A). In contrast, bees in the Rut (z=−2.097, q=0.077) and Rut+Fipro (z=−0.136, q=0.42) group exhibited learning scores that were not significantly different from Control bees (FIG. 4A). These results are strong evidence of the impairment on learning produced by imidacloprid and fipronil and the innocuous effect of rutin. Moreover, the response of Rut+Fipro bees demonstrate full protection by the flavonoid. Importantly, the significant difference between Rut+Imid and Control bees indicated a lack of protection. However, a post-hoc comparison showed that bees in the Rut+Imid group exhibited significantly higher scores than Imid bees (z=4.2, p<0.0001). Thus, prophylactic administration of rutin partially, but significantly, protected against the effect of imidacloprid while providing full protection against fipronil.

In contrast to the results about learning, differences in latency of response in bees was not observed for any treatment relative to Control bees (Dunnet's test: p>0.05 for all treatments). However, it was found that treatments significantly impacted the decision-making process, assessed as the ratio between latency of response and learning score (ANOVA: F_{5,122}: 5.477, p=0.001). Relative to Control bees, a significant increase in the decision-making ratio of Imid (t_{1,140}: 4.226, q=0.0001), Fipro (Student-t test: t_{1,47}: 2.148, q=0.0166) and Rutin+Imid bees (Student-t test: t_{1,47}: 3.266, q=0.0480) was found.

After 24 h, a significant effect of the administered treatment on memory retention (Likelihood ratio: Chi-Square: 18.78, p=0.0021) was found. Only 13% of the bees receiving imidacloprid and 34.6% of the bees receiving fipronil exhibited a conditioned response, in contrast to 64.3% of the Control bees, thus providing strong evidence that the administration of the insecticides significantly decreases the probability of remembering (Adjusted Wald test for two proportions for a PER response: proportion difference Control-Imid: 0.51, q=0.0003; proportion difference Control-fipro: 0.30, q=0,016; FIG. 4B). The odds of remembering were estimated to be 12 times larger for Control bees relative to Imid bees and 3.4 times larger relative to Fipro bees.

Importantly, 42.9% of the bees in the Rutin+Imid group and 60% of the bees in the Rut+Fipro group remembered after 24 h, thus providing strong evidence of the protective effect on memory of the flavonoid relative to Imid and Fipro bees (Adjusted Wald test for two proportions: proportion difference Imid-Rutin+Imid for a PER response: −0.30, q=0.016; proportion difference Fipro-Rutin+Fipro: −0.25, q=0.028; FIG. 4B). The odds of remembering were estimated to be 5 times larger for Rut+Imid bees relative to Imid bees and 2.8 times larger for Rut+Fipro relative to Fipro bees. Together, these results are strong evidence that ad libitum administration of the flavonoid Rutin protects against the cognitive impairments on learning, memory and decision-making of two insecticides with different pharmacodynamics. Imidacloprid targets primarily nAChR whereas Fipronil targets GABAergic and dopaminergic receptors.

### Example 5: Flavonoids Administered by a Single Day Protect Honey Bee Sensory Sensitivity Against a Pyrethroid-Induced Impairment

### Materials and Methods

Honey Bee Collection and Maintenance

Honey bee foragers (Apis mellifera scutellata) were captured from established hives at the Apiary of the Universidad del Rosario (Bogota, Colombia) between 08:00 h and 10:00 h. Bees were ice-anesthetized and harnessed in plastic tubes and maintained under laboratory conditions for the duration of the experiment (three days).

Experimental Treatments

On the first day, bees were randomly allocated to one of four treatments administered as two doses (morning: 20 μL; afternoon:10 μL): i) Control (1M sucrose solution), ii) Rutin 1 μM (Sigma-Aldrich R5143), iii) Kaempferol 131.7 μM (Sigma-Aldrich K0133), iv) Rutin-Kaempferol composition (final concentration of 1p M rutin and 131.7 μM kaempferol). All solutions were diluted in 1M sucrose solution to the concentrations described above. The next day, bees from each group were randomly assigned to one of two treatments: 5 NL of 1M sucrose solution or 17.81 μM deltamethrin (Dinastia Bayer). Thus, bees belonged to one of eight treatments: i) Sucrose water (Control), ii) Rutin (Rut), iii) Kaempferol (Kaemp), iv) Kaempferol+Rutin (Kaemp+Rut), v) Deltamethrin (Delta), vi) Rutin+Deltamethrin (Rut+Delta), vii) Kaempferol+Deltamethrin (Kaemp+Delta), viii) Kaempferol+Rutin+Deltamethrin ((Kaemp+Rut)+Delta). All solutions were diluted in 1M sugar water to the concentrations described above.

Sucrose Responsiveness of Individual Honey Bees

Sensory sensitivity was evaluated using responsiveness to sucrose, a proxy of resource exploitation by foragers. On the second day, bees of the eight treatments described above were tested five times for their response threshold (0 h, 1 h, 2 h, 3 h and 4 h). The first measure (0 h) was done prior to pesticide administration. Bees were tested with an ascending sucrose concentration of 0.1, 0.3, 1, 3, 10 and 30% (w/w). Between each concentration, the bees were presented with pure water as a control for potential sensitization or habituation (Pankiw et al., Behav Ecol Sociobiol (2000) 47:265-267). The inter-trial time between the presentation of each sucrose concentration was at least 4 minutes. For each presentation, the reflexive extension of the proboscis (tongue) of the bee, after antennal stimulation with the solution was recorded. After the last measure (4 h), bees were fed with 10 μL of 1M sucrose solution and maintained until the next day for a final test (24 hours after pesticide administration).

Data Collection and Statistical Analysis

For each bee, a responsiveness score was determined (0-7) 0 h, 1 h, 2 h, 3 h, 4 h and 24 h, and a pooled scored (0-42). Normal distribution of the scores was tested using Shapiro-Wilk W Test for the null hypothesis that data is from a Normal distribution. The score across treatments did not exhibit a normal distribution (Control: W=0.824, p<0.0001; Ka: W=0.821, p<0.0001; Rut: W=0.672, p<0.0001; Kaemp+Rut: W=0.817, p<0.0001; Delta: W=0.944, p=0.024; Kaemp+Delta: W=0.94, p=0.0154; Rut+Delta: W=0.929, p=0.009; Kaemp+Rut+Delta: W=0.91, p=0.0008) and could not be normalized using transformation. Thus, non-parametric tests were used for comparisons across treatments and for multiple comparisons. Overall changes of responses across different concentrations were evaluated using repeated-measured MANOVA. Error associated with multiple comparisons was controlled using the Two-stage sharpened False Discovery Rate, and corrected p-values (q-values) are presented. Data were explored for outliers using a single run of Robust Fit outliers test. All analyses were conducted using JMP v.14 (SAS Institute Inc.)

### Results

The protective effect of two flavonols (rutin and kaempferol), and a composition with their mixture, against the negative sublethal sensory effects of the pyrethroid deltamethrin was investigated using sucrose responsiveness. Following the outliers screening, 70 bees were excluded, and final analyses was run on 402 bees distributed across eight treatments: Control (N=50), Kaempferol (N=50), Rut (N=56), Kaemp+Rut=52), Delta (N=48), Kaemp+Delta=49), Rut+Delta (N=45), (Kaemp+Rut)+Delta (N=52).

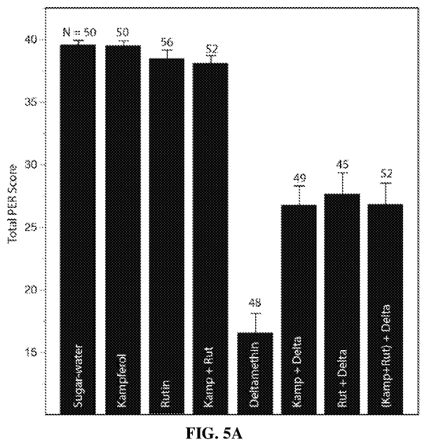

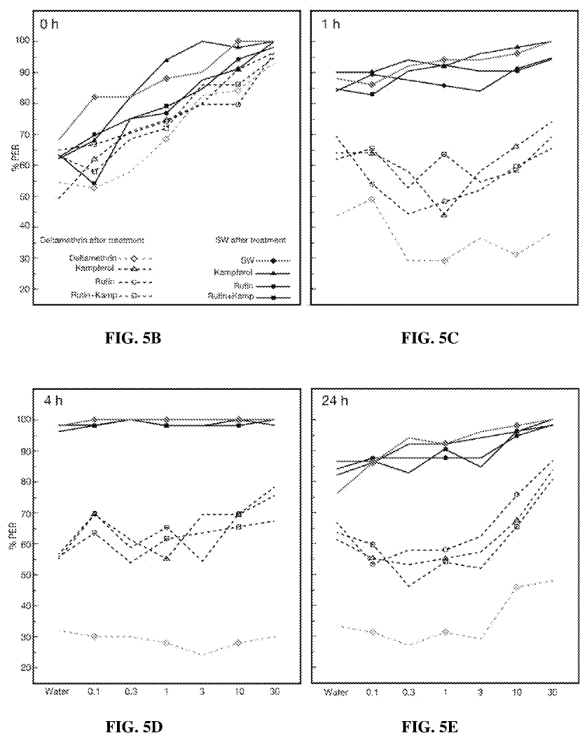

Results indicated that exposition to deltamethrin had a significant detrimental effect on the sucrose responsiveness. Bees' sensitivity was significantly affected by treatment (Kruskal-Wallis: X7=168.87, p<0.0001). Relative to Control bees (FIG. 5A), and the sensitivity was significantly lower in Delta (z=7.85, q<0.0001), Kaemp+Delta (z=6.38, q<0.0001), Rut+Delta (z=5.31, q<0.0001) and (Kaemp+Rut)+Delta bees (z=5.96, q<0.0001). In contrast, relative to Control bees, sensitivity was not significantly affected in Kaemp (z=0.05, q=0.45), Rut (z=−0.01, q=0.45) and Kaemp+Rut bees (z=1.10, q=0.45). These results demonstrated the impairment on sensory sensitivity produced by the pyrethroid and not the innocuous effect of the flavonoids. Remarkably, following post hoc comparisons relative to Delta bees, a significantly higher sensitivity in Kaemp+Delta (z=−4.11, q<0.0001), Rut+Delta (z=4.24, q<0.0001) and (Kaemp+Rut)+Delta bees (z=−4.02, q=0.0001) was found. These results demonstrate the significant protection provided by the flavonoids.

Regarding sensitivity across time, for all treatments, at baseline (0 h), responsiveness was significantly affected by concentration of sucrose water (F_{6,389}: 46.911, p<0.0001). A stimulation with a higher concentration of sucrose water led to a higher percentage of bees exhibiting the reflexive response (FIG. 5B). This result reflects the normal responsiveness of bees when encountered with resources of varying concentration. Interestingly, deltamethrin had a rapid and lasting negative effect on the sucrose response. After the administration of the insecticide, three groups can be differentiated (FIG. 5B for examples at 1 h, 4 h (last measure of the day) and 24 h). Bees receiving deltamethrin exhibited the lowest sensitivity across all testing times, including 24 h after administration of the insecticide relative to Control bees (Delta: z=6.95, q=0.0002; Kaemp+Delta: z=4.28, q=0.0002; Rut+Delta: z=3.44, q=0.003; (Kaemp+Rut)+Delta: z=4.21, q=0.0002). Remarkably, relative to Delta bees, administration of a flavonoid or the composition followed by exposure to the insecticide led to significantly higher sensitivities after 24 h (Kaemp+Delta: z=−3.71, q=0.0006; Rut+Delta: z=3.78, q=0.0006; (Kaemp+Rut)+Delta: z=−3.19, q=0.0019).

The decrease in sensitivity observed after the administration of deltamethrin affected the response across all concentrations of sucrose solution (FIG. 5B). After one hour, less than 40% of bees exposed to deltamethrin were responsive to 30% sucrose-water, the highest concentration used for the test. After 4 hours, less than 30% of bees responded to 30% sucrose water and after 24 hours an improvement of merely 10% was observed. In contrast, bees in the Rut+Delta, Kaemp+Delta or (Kaemp+Rut)+Delta treatments never exhibited less than 60% of responsiveness to 30% sucrose-water and after 24 h exhibited at least 80% responsiveness to 30% sucrose-water. In fact, the odds of responding to 30% sucrose water relative to Delta bees were estimated to be 5.6 times higher for Kaemp+Delta bees, 7.1 times higher for Rut+Delta bees and 4.6 times higher for (Kaemp+Rut)+Delta bees.

Hence, these results demonstrate the protective effect of the prophylactic administration of the flavonols rutin and kaempferol, as well as their composition, against the sensory impairment induced by the exposition to the pyrethroid deltamethrin. Remarkably, these results demonstrate a protective effect that lasted over 36 hours after the last dose of a flavonoid provided for a single day. This indicates that the protection is, at least partially, due to long lasting mechanisms, such as induction of CYP450.

Unlike neonicotinoids, which target specific nAChR, pyrethroids such as deltamethrin target primarily the voltage dependent sodium channels (Na⁺ᵥ), broadly present across excitable tissues (mostly muscles and neurons). Hence, this broader impact should affect motor, sensory and cognitive systems. The results herein indicate motor and sensory impairments, reflected as slow PER and responses only to high concentrations of sucrose solution. While pyrethroids have been proposed as an alternative to the banned neonicotinoids, these results show that the ingestion of deltamethrin lead to negative impacts that may extend to the energy acquisition of the colony. Moreover, together the results show that the protection offered by flavonoids extends to pesticides with different mechanisms of action.

### Example 6: Flavonoids Rutin and Kaempferol and the Phenolic Acid p-Coumaric Acid Protect Honey Bees Against Impairment Induced by Administration of Fipronil

### Materials and Methods

Honey Bee Collection and Maintenance

Honey bee foragers (Apis mellifera scutellata) were captured from established hives at the Apiary of the Universidad del Rosario (Bogota, Colombia) between 08:00 h and 10:00 h. Bees were ice-anesthetized and harnessed in plastic tubes and maintained under laboratory conditions for the duration of the experiment (five days). Food was provided twice a day in a volumen of 10 μL and varied in composition according to experimental treatments.

Experimental Treatments

Bees were randomly allocated to one of the following treatments administered twice a day for three consecutive days (all concentrations are final):

Control: 1M sucrose water (10 μL)

Rut: 1 μM rutin (Sigma-Aldrich: R5143)-1M sugar water (10 μL)

Kaemp:131 μM kaempferol (Sigma-Aldrich:K0133)+1M sugar water (10 μL)

p-Cou A: 200 μM p-coumaric acid (Sigma-Aldrich:C9008) diluted in 1M sugar water (10 μL)

Mix: 1 μM rutin, 131 μM kaempferol and 200 μM p-Coumaric acid): Bees fed with composition diluted in 1M sugar water (10 μL) fed twice a day for three days

On the fourth day, bees were randomly assigned to one of two treatments three hours before training:

Fipronil: 10 μL of 228 nM Fipronil (Astuto Invesa) in 1M sucrose water

Sucrose solution: 10 μL 1M sucrose water

Thus, bees belong to one of ten treatments: Control, Kaemp, Rut, p-Cou A, Mix, Fipro, Kaemp+Fipro, Rut+Fipro, p-Cou A+Fipro, Mix+Fipro.

Training

Bees were trained using classical conditioning of the proboscis extension response (PER). Briefly, bees received a soft current of clean air for 15 seconds to allow bees to accommodate. Then a soft current of 1-hexanol (Sigma-Aldrich: 471402) was injected for 10 seconds. Six seconds after injection of 1-hexanol, one of the antennae was stimulated with sugar water (1.5M), which leads to the reflexive extension of the proboscis, and the bee was allowed to drink for 4 seconds. Thus, the reward and the stimulus overlapped for four seconds. This sequence, recognized as a training trial, was repeated eleven times. Time was determined using a metronome (2 Hz). Memory retention was tested 24 hours after the eleven training trials. During each trial, whether the bee exhibited a conditioned PER was recorded. 12 bees were trained each session. Training was conducted by an experimenter blind to treatment.

Data Collection and Statistical Analysis

Learning for each individual was quantified as the number of conditioned PER responses across the trials. Thus, for each bee a learning score between zero and ten (0-10) was recorded. The scores across the six treatments were compared using a Kruskal-Wallis test since the data did not follow a normal distribution and could not be normalized after transformations.

Multiple comparisons were then run comparing learning score relative to Control bees using a non-parametric one or two-tailed Steel Method depending upon predictions. These comparisons informed us about the predicted impairment produced by the insecticides, the predicted innocuous effect of the flavonoid and the potential level of protection. Error due to multiple comparisons was controlled using the False Discovery Rate method (FDR-two stage sharpened method). In all cases corrected p-values (q-values) are presented.

Memory retention was quantified as a nominal variable (yes/no). Data were compared across treatments using a Chi-square test, followed by multiple comparisons of all treatments vs. Control. Error due to multiple comparisons was controlled using the FDR as indicated above. In all cases corrected p-values (q values) are presented.

Latency was quantified as a continuous variable between 0.5 s and 10 s. Data were compared across treatments using an ANOVA following a Log transformation to normalize the distribution (Shapiro-Wilk W Test after log transformation: p>0.05 for all treatments for the null hypothesis that data is from a normal distribution), followed by multiple comparisons between all treatments and the Control group (Dunnett's method). Error due to multiple comparisons was controlled using the FDR method as indicated above. In all cases corrected p-values (q-values) are presented.

### Results

Experiments were designed to evaluate the impairing effect of fipronil and the protective effect of the flavonoids kaempferol and rutin and the phenol p-coumaric acid in honey bees.

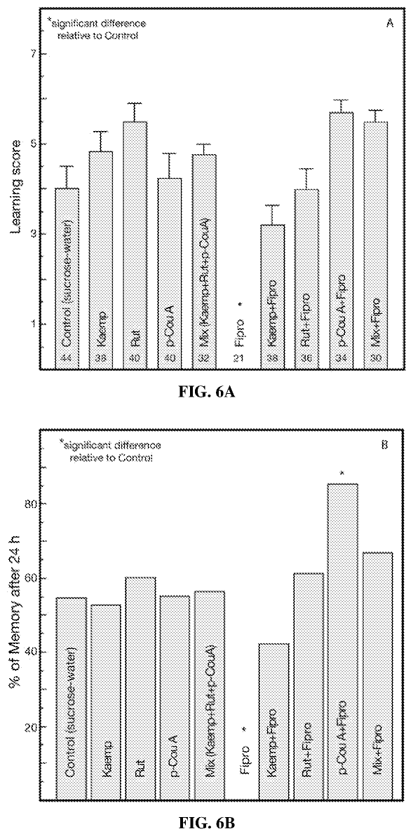

The data for outliers was analyzed using Mahalanobis distances including the learning score and memory retention as variables. 41 bees were excluded and final analyses were conducted on 353 bees across the 10 treatments (FIG. 6): Control (N=44), Rut (N=40), Kaemp (N=38), p-CouA (N=40), Mix (N=32), Fipro (N=21), Rut+Fipro (N=36), Kaemp+Fipro (N=38), p-CouA+Fipro (N=34), Mix+Fipro (N=30).

Results show that learning score was significantly affected by treatment (Kruskal-Wallis test: X₉=65.97, p<0.0001; FIG. 6). Bees in the Fipro group exhibited learning scores significantly lower than Control bees (z=5.176, q=0.00042). Bees in all other groups exhibited learning scores that were not significantly different from Control bees (Rut: z=−2.179, q=0,526; Kaemp: z=−1.235, q=0,933; p-CouA: z=−0.0159, q=0,933; Mix: z=−1.218, q=0.933; Rut+Fipro: z=0.005, q=0.933; Kaemp+Fipro: z=1.121, q=0.933; p-CouA+Fipro: z=−2.318, q=0.933; Mix+Fipro: z=−1.975, q=0.284). These results are strong evidence of the impairment on learning produced by fipronil and the innocuous effect of rutin, kaempferol and p-Coumaric acid. Moreover, the response of Rut+Fipro, Kaemp+Fipro, p-CouA+Fipro and Mix+Fipro bees demonstrate full protection by the flavonoids and the phenolic acid.

Further, an overall analysis of latency of response indicated a significant effect of treatment (Kruskal-Wallis test: X₈=23.137, p=0.0032). Bees in the Kaemp+Fipro group exhibited significantly longer latencies than Control bees (Dunnett's method: LSD=0.045, q=0.009). Bees in all other groups exhibited latencies that were not significantly different from Control (Dunnett's method: Rut: LSD=−0.06, q=0.919; Kaemp: LSD=−0.01, q=0.232; p-CouA: LSD=−0.01, q=0.232; Mix: LSD=−0.06, q=0.919; Rut+Fipro: LSD=−0.09, q=0.919; p-CouA+Fipro: LSD=−0.03, q=0.498; Mix+Fipro: LSD=−0.01, q=0.232).

After 24 h, a significant effect of the administered treatment on memory retention (Likelihood ratio: Chi-Square: 53.09, p<0.0001; FIG. 6B) was found. None of the bees receiving fipronil exhibited a conditioned response, in contrast to 54.6% of the Control bees, thus providing strong evidence that the administration of the insecticide significantly decreases the probability of remembering (Adjusted Wald test for two proportions for a PER response: proportion difference Control-Fipro: 0.545, q=0.0004).

Remarkably, 85.3% of the bees in the p-CouA+Fipro group, 42.1% in the Kaemp+Fipro group, 61.1% in the Rut+Fipro group and 56.2% in the Mix+Fipro group exhibited a conditioned response after 24 h thus providing strong evidence of the protective effect on memory of the flavonoids and the p-coumaric acid. Interestingly, bees in p-CouA+Fipro group exhibited a significantly higher probability of exhibiting a conditioned response than Control bees (Adjusted Wald test for two proportions: proportion difference Control-p-CouA+Fipro for a PER response: 0.31, q=0.01). The odds of remembering were estimated to be 4.8 times larger for p-CouA+Fipro bees relative to Control bees.

Together, these results are strong evidence that administration of the flavonoids rutin and kaempferol and the phenolic acid p-coumaric acid protect against the cognitive impairments on learning and memory induced by the administration of fipronil.

### Example 7: Ad Libitum Self-Administration of a Flavonoid Protects Bumblebee Learning and Memory Against Chronic Administration of Fipronil

Materials and Methods

Bumblebee Collection, Maintenance and Experimental Treatments

One colony of the bumblebee Bombus impatiens (Koppert Inc) was kept in laboratory conditions. Bees received pollen inside the nests and foraged to a 1M sugar-water feeder outside of the nest. Bees were collected while on the feeder and iced anesthetized to then be randomly assigned to one of four plastic containers where self-administration of one of two solutions was allowed to 10 bees out of two feeders (glass vials with 500 μL of solution each): i) 1 M sucrose water or ii) 1 μM Rutin (Sigma-Aldrich R5143) diluted in 1M sucrose water. Feeders were refilled each day for three days. On the night of the third day, one of the vials was replaced by a new one with one of two solutions: i) 1 M sucrose water or ii) 1M sucrose water+6 nM fipronil. Feeders were refilled each day for three more days, thus providing a chronic exposure to fipronil. On the night of the third day, bees were iced anesthetized and yolked restrained in plastic pipettes. Thus, bees belonged to one of four treatments:

Control: self-administered sucrose water for six days

Rut: self-administered rutin for six days

Fipro: self-administered sucrose water for three days followed by three days of self-administered sucrose water (one feeder) and fipronil (one feeder).

Rut+Fipro: self-administered rutin for three days followed by three days of self-administered rutin (one feeder) and fipronil (one feeder).

Training

All bees were blind-trained using olfactory conditioning of the proboscis extension response (PER). Briefly, bees were exposed to a presentation of 1-nonanol (Sigma-Aldrich) for 10 s. After 7 s of the onset of the odor presentation, the antenna was stimulated with 1.5 M sugar-water to elicit the PER. Following the PER, the bee was allowed to drink sugar-water for 3 s. Each paired presentation was considered a training trial and each individual received eleven training trials with an average intertrial interval of 10 minutes. After training, all bees were fed 20 μL of 1M sugar water and maintained in plastic boxes for 24 h until the retention test. For each trial whether a conditioned PER was exhibited and its latency in seconds using a metronome (2 Hz) was recorded. Twenty-four hours after the last training trial the bees were presented with the conditioned odor for 10 s. Whether a bee exhibited a conditioned PER was recorded. Bees not exhibiting a conditioned PER were stimulated with sucrose water in order to test motivation.

Data Collection and Statistical Analysis

Learning for each individual was quantified as the number of conditioned PER responses across the trials. Thus, for each bee a learning score between zero and ten (0-10) was recorded. The scores across the six treatments were compared using a Kruskal-Wallis test since the data did not follow a normal distribution and could not be normalized after transformations.

Multiple comparisons were then run comparing learning score relative to Control bees using a non-parametric one or two-tailed Steel Method depending upon predictions. These comparisons informed us about the predicted impairment produced by the fipronil, the predicted innocuous effect of the rutin and the potential level of protection. Error due to multiple comparisons was controlled using the False Discovery Rate method (FDR-two stage sharpened method). In all cases corrected p-values (q-values) are presented.

Memory retention was quantified as a nominal variable (yes/no). Data were compared across treatments using a Chi-square test, followed by multiple comparisons of all treatments vs. Control. Error due to multiple comparisons was controlled using the FDR as indicated above. In all cases corrected p-values (q values) are presented.

Latency was quantified as a continuous variable between 0.5 s and 10 s. Data were compared across treatments using an ANOVA following a Log transformation to normalize the distribution (Shapiro-Wilk W Test after log transformation: p>0.05 for all treatments for the null hypothesis that data is from a normal distribution), followed by multiple comparisons between all treatments and the Control group. Error due to multiple comparisons was controlled using the FDR method as indicated above. In all cases corrected p-values (q-values) are presented.

### Results

The protective effect of the flavonol rutin against the negative sublethal effects of chronic exposure to fipronil was investigated using the conditioning of the proboscis extension response.

Data for outliers was analyzed using Mahalanobis distances including the learning score and memory retention as variables. 24 bees were excluded and final analyses were conducted on 75 bees distributed across the four treatments: Control (N=16), Rut (N=20), Fipro (N=24), Rut+Fipro (N=15).

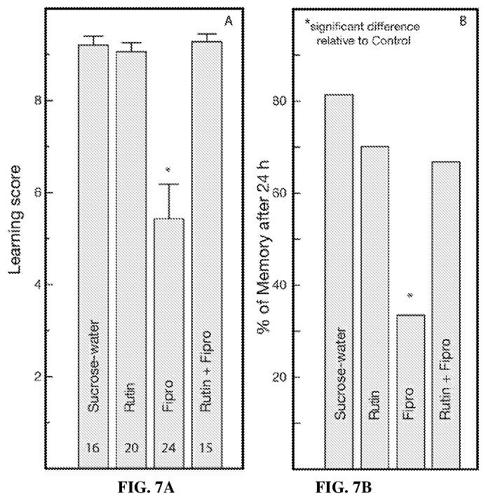

Results show that learning score was significantly affected by treatment (Kruskal-Wallis test: X₃=18.145, p=0.0004). Bees in the Fipro group exhibited a learning score significantly lower than Control bees (z=−3.19, q=0.006; FIG. 7A). In contrast, bees in the Rut (z=−0.363, q=0.699) and Rut+Fipro (z=0.109, q=0.699) groups exhibited learning scores that were not significantly different from Control bees (FIG. 7A). These results are strong evidence of the impairment on learning produced by fipronil and the innocuous effect of rutin. Moreover, the response of Rut+Fipro bees demonstrate full protection by the flavonoid. Moreover, a post-hoc comparison showed that bees in the Rut+Fipro group exhibited significantly higher scores than Fipro bees (z=3.198, p=0.0028). Thus, prophylactic self-administration of rutin fully protected against the effect of chronic administration of fipronil.

After 24 h, a significant effect of the administered treatment on memory retention (Likelihood ratio: Chi-Square: 11.43, p=0.0096) was found. Only 33.3% of the bees receiving fipronil exhibited a conditioned response, in contrast to 81.2% of the Control bees, thus providing strong evidence that the administration of this insecticide significantly decreases the probability of remembering (Adjusted Wald test for two proportions for a PER response: proportion difference Control-Fipro: 0.48, q=0.0007; FIG. 7B). The odds of remembering were estimated to be 8.7 times larger for Control bees relative to Fipro bees.

Most importantly, 66.7% of the bees in the Rut+Fipro group remembered after 24 h, thus providing strong evidence of the protective effect on memory of the flavonoid relative to Fipro bees (Adjusted Wald test for two proportions: proportion difference Fipro-Rut+Fipro for a PER response: −0.33, q=0.0108 FIG. 7B). The odds of remembering were estimated to be 4 times larger for Rut+Fipro bees relative to Fipro bees. Together, these results are strong evidence that self-administration of the flavonoid rutin protects against the cognitive impairments on learning and memory of fipronil.

### Example 8: Flavonoid Protects Bees by Stabilizing the nACh Receptor in a Wider Range of Conformational States than Imidacloprid and Closer to the Natural Agonist for Shorter Periods of Time

### Materials and Methods

Creating 3D Model for Nicotinic Acetylcholine Receptor of Apis mellifera from 3D Crystallographic Model from Apis mellifera.

In order to model A. mellifera nAChR alpha6, the aminoacid sequence was query from NCBI database (Elisk et al, BMC genomics, 15, 86. 2014) and protein blast was made against ProteinDataBank (pdb) database to find the closest proteins with a crystalline structure to use as template. A. mellifera's protein sequence was aligned using MUSCLE to each sequence individually. MODELLER 9.23 was used through PyMod PyMol plugin to build a protein template complex and model the tridimensional structure of A. mellifera's nAChR alpha6 pentamere (Fisher & Sali. In Methods in Enzymology, C. W. Carter and R. M. Sweet, eds. Academic Press, San Diego, 374, 463-493, 2003; Janson et al. Bioinformatics (Oxford, England), 33(3), 444-446. 2017). Five models were calculated and the one with the lowest DOPE score was used Loop. Refinement algorithms were applied to refine gaps in the model.

Molecular Docking

Acetylcholine (ACh), Imidacloprid (IMI), Quercetin (Quer), 4′-Phenylflavone and Rutin (Rut) were docked into their ACh and IMI reported binding site (Colquhoun & Silvilotti. Trends in Neurosciences, 27(6), 337-344. 2004; Le Questel et al. Journal of Molecular Graphics and Modelling, 55, 1-12. 2015). The interaction between ACh, IMI and flavonoids with nAChR was analysed and compared. Molecular docking (Autodock4 and Autodock Tools; Morris et al., Journal of Computational Chemistry 2009, 16: 2785-91. 2009) was conducted between each of the ligands and the receptor, and determined the minimal conformation energy (Avogadro 1.2.0 with Open Babel 2.3.90). Force field MMFF94 with steepest descent and 2 steps per update was used as forcefield avoiding local energy overestimation (Halgren. Journal of Computational Chemistry, 20: 720-729. 1999). In order to prepare the ligand and receptor for the molecular docking the atoms coordinates of the optimized ligands were locked to avoid ligand movement. Complex files were exported in PDB format.

Molecular Dynamics

Trajectory of nAChR state was simulated by creating a GROMACS topology from each ligand-receptor complex PDBs and creating an general AMBER force field (GAFF) with prior whole system energy minimization. Then, the complex system was fixed in order to create an octahedron solvent box that was filled with water molecules. Protein was centred with a maximum distance of 0.8 nm between it and the box farthest boundary. Ion concentration was fixed at 423 mOsm to match ionic concentration in bee hemolymph according to Leonhard, B., & Crailsheim (Amino Acids, 17(2), 195-205. 1999). This complex system energy was again minimized to −500 kJ/mol/nm as indicated by (Berendsen, der Spoel, & Drunen. Computer Physics Communications, 91(1), 43-56. 1995) and equilibrated to normal conditions (298,37 K and 1 bar). The simulation was run for 2 microseconds. BioExcel Building Blocks (BioBB) library for python 3.7 was used to execute all previous procedures (Andrio et al., Scientific Data, 6(1), 169. 2019).

Trajectory Analysis

The trajectory of all ligand-receptor complex was compared by performing dimensional reduction of trajectory data via principal component analysis. The summary of the different trajectories and the time spent in each state during the time of the simulation was obtained from principal components (PC) which cumulative variance was above 85% (regularly up to the fifth PC). An active conformational state was identified where the Acetylcholine-receptor complex spent the largest amount of time and where pore radius was larger (Dror et al., Chemical Reviews, 117(1), 139-155. 2017). Trajectory analysis was performed using MDAnalysis library for Python 3.7 (Beckstein et al., Proceedings of the 15th Python in Science Conference, pages 98-105, Austin, Tex., 2016. SciPy. 2017; Beckstein et al., Journal of Computational Chemistry. 32, 2319-2327. 2011). Conformational states were plotted for comparison using VMD v.1.9.3 (Humphrey, Dalke, & Schulten. Journal of Molecular Graphics, 14, 33-38. 1996).

Pore Channel Analysis

A pore channel analysis was conducted on nAChR using HOLE program (Smart et al., Biophysical Journal. 65(6): 2455-2460. 1993) through MDAnalysis library, in order to assess the physiological significance of the interaction between nAChR and the ligands. Conformational states associated to larger pore diameter were labelled as active state of the channel (i.e. increased ion permeability). The analysis was performed on the steps files from the trajectory analysis.

Statistical Analysis

A two sample Kolmogorov-Smirnov test was used to evaluate differences in the distribution of the time spent in the range of conformational states recorded in the first PC. Significance was evaluated at alpha=0.05. The analysis was run using scikitlearn library for python 3.7 (Duschesnay et al. Journal of Machine Learning Research. 12, 2825-2830. 2011)

### Results

Following comparison, it was found that statistical differences between the time spent in the range of conformational states when nAChR is coupled with Imidacloprid, Acetylcholine and the flavonoids (Table 3).

TABLE 3
Two sample Kolmogorov-Smirnov test results from
the comparison of conformational states distribution
of nACHr when coupled to different ligands.

| Comparison | K statistic | P value |
|---|---|---|
| Imidacloprid-Acetylcholine | 0.5523 | 8.49e−30 |
| Imidacloprid-Quercetin | 0.2190 | 7.94e−5 |
| Imidacloprid-Rutin | 0.5142 | 1.12e−019 |
| Imidacloprid-Rutin | 0.2809 | 1.05e−7 |
| Acetylcholine-Quercetin | 0.3666 | 6.02e−13 |
| Acetylcholine-Rutin | 0.1476 | 0.0204 |
| Acetylcholine-4′Phenylflavone | 0.36666 | 6.02E−13 |

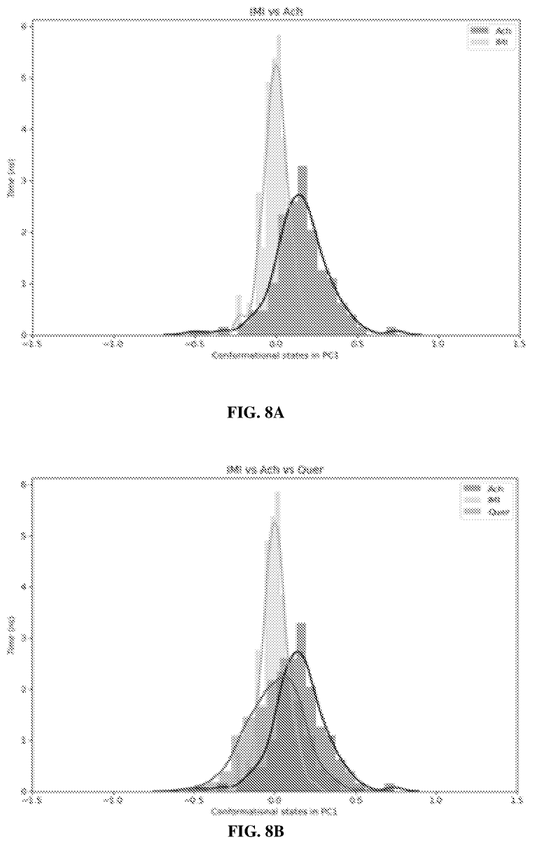

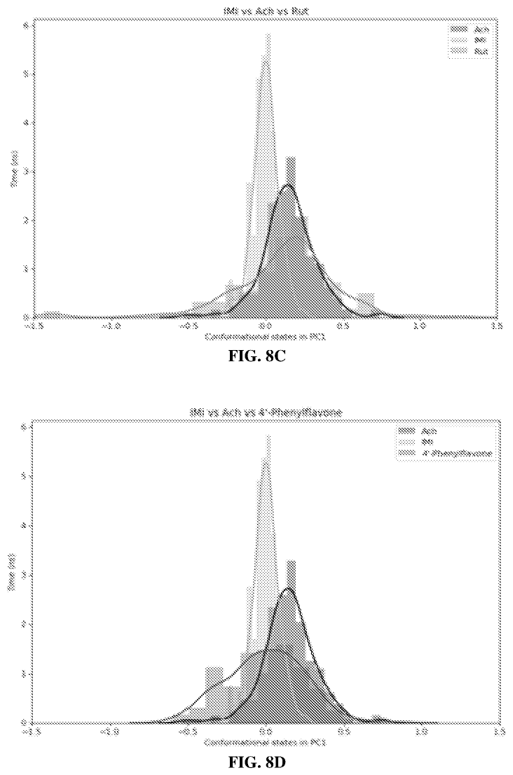

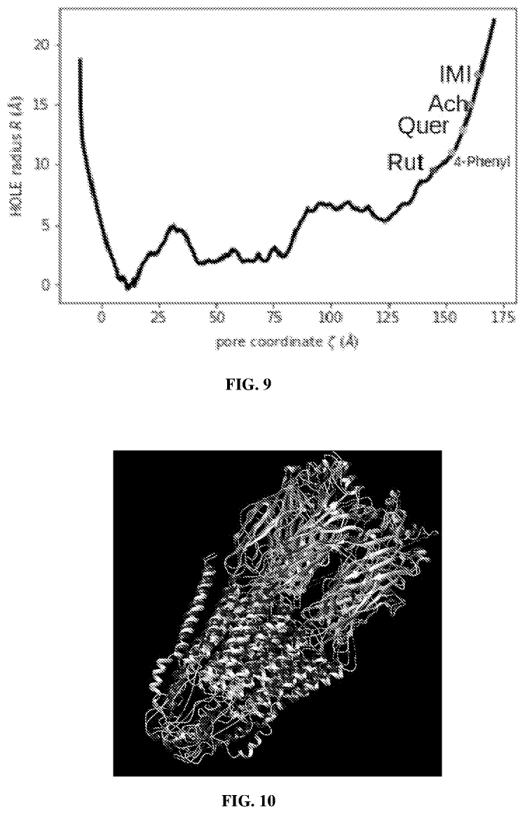

Principal component 1 represented 68% of variance from the trajectories. The protein shows a significant difference in the conformational states distribution in time when exposed to the natural agonist (ACh) and any of the agonists (FIG. 1). Both Imidacloprid (IMI) and the flavonoids stabilize receptor conformational state closely overlapping to that of the natural agonist. However, their position is displaced toward the inactive state. Despite being conformationally close, IMI fixes the receptor into a more stable and lasting position than flavonoids or the natural agonist (FIG. 8 A-D). This is an indicator of the toxic effect of IMI via overstimulation of the receptor. The frequency distribution of IMI is statistically different to that from all flavonoids and the agonist (Table 3). Hence, the three molecules produce a similar excitatory effect. Despite being significantly different in their distribution, the effect of Quercetin is the most similar to that of Acetylcholine (FIG. 8B) for the conformational state and the time spent in active positions. The effect of Rutin and 4′-Phenylflavone is a more disperse set of conformational states in a shorter period of time (FIGS. 8C and 8D).

These results are believe to indicate that the natural agonist exerts its physiological function by displacing the receptor to a suboptimal position during a short period while IMI is able to displace it onto a maximum activity related position for a longer period (FIG. 10). Flavonoids occupy an intermediate spatial and temporal states. Hence, they interact/bind to the receptor with a lower probability of opening the channel. Because flavonoids bind to the same site than IMI, the overstimulation of the receptor is prevented by flavonoids competing for the binding without producing the same degree of activation.

These findings are consistent with evidence of IMI acting as agonist (Decourtye et al. Pesticide Biochemistry and Physiology, 78(2), 83-92. 2004) or partial agonist (Déglise, Grunewald, & Gauthier. Neuroscience Letters, 321(1), 13-16. 2002). They also explain the neuroprotective effects of some flavonoids against the cognitive impairment developed by bees when exposed to neonicotinoids (see, e.g., Examples above), when a competitive binding interaction is contemplated (Wong, Liao, & Berenbaum. PLoS ONE, 13(11), 1-15. 2018). Moreover, a similar pattern is feasible on different levels such as mitochondrion interaction with both molecules. In summary, these findings provide a mechanistic effect of the dose dependent protective effect of the mentioned flavonoids. Flavonoids with similar structural characteristics are believed to provide the observed effect of Quercetin, Rutin or 4′-Phenylflavone, depending on their level of similarity; therefore, the results are believed to be extendable to such molecules.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of protecting insect pollinators from a pesticide, the method comprising administering to insect pollinators a composition comprising an effective amount of one or more phenolic compounds to prevent or reduce impairment of a cognitive function and/or increase a cognitive function in the insect pollinator following exposure to the pesticide.

2. The method of claim 1, wherein the one or more phenolic compounds is one or more flavonoids, p-coumaric acid, or a combination thereof.

3. The method of claim 2, wherein the flavonoid is selected from the group comprising flavones, flavanones, flavonols, isoflavones, anthocyanins, flavanols (catechins), chalcones, and neoflavonoids.

4. The method of claim 3, wherein the flavonoid is a favonol.

5. The method of claim 1, wherein the one or more phenolic compounds is selected from quercetin, rutin, myricetin, kaempferol, fisetin, morin, isorhamnetin, p-coumaric acid, and derivatives or variants thereof, and combinations thereof.

6. The method of claim 1, wherein the one or more phenolic compounds is selected from the compounds of Table 1 (e.g., isoquercetin, rutin, quercetin, quercitrin, hyperoside, quercimeritrin, baimaside, querciturone, 3-O-methylquercetin, quercetin 3-sambubioside, miquelianin, spiraeoside, isorhamnetin, quercetin 3-O-sulfate, quercetin 7-O-glucoside, quercetin 3-O-Rhamnoside, quercetin 3,4′-diglucoside, and quercetin 3-sophorotrioside) and combinations thereof.

7. The method of claim 5, wherein an area occupied by the insect pollinators has been or will be treated with a pesticide.

8. The method of claim 7, wherein the area occupied by the insect pollinators is also occupied with insect pests.

9. The method of claim 8, further comprising treating the area occupied by the insect pollinators with the pesticide.

10. The method of claim 9, wherein the area occupied by the insect pollinators is treated with the pesticide before the composition is administered to the insect pollinators.

11. The method of claim 9, wherein the area occupied by the insect pollinators is treated with the pesticide after the composition is administered to the insect pollinators.

12. The method of claim 10 or 11, wherein the insect pollinators are administered the composition in the same area treated with the pesticide.

13. The method of claim 10 or 11, wherein the insect pollinators are administered the composition in an area separate from the pesticide.

14. The method of claim 10 or 11, wherein the area is treated with an effective amount of pesticide to reduce the number of insect pests in the area.

15. The method of claim 14, wherein the pesticide is lethal to insect pests.

16. The method of claim 14, wherein the area is treated with an amount of pesticide that is sublethal to insect pollinators.

17. The method of claim 16, wherein the insect pests are selected from the group consisting of aphids, mites, thrips, whiteflies, leafhoppers, mealybugs, spittlebugs, fleas, termites, scales, beetles, and combinations thereof.

18. The method of claim 17, wherein the insect pollinators are selected from butterflies, moths, flies, beetles, wasps, bees, and combination thereof.

19. The method of claim 18, wherein the bees are selected from honeybees, bumblebees, carpenter bees, leafcutter bees, blueberry bees, squash bees, mason bees, stingless bees, orchid bees, sweat bees, and combinations thereof.

20. The method of claim 19, wherein the cognitive function is selected from learning, memory, attention, decision accuracy, decision speed, navigation, motor activity, sucrose sensitivity, and combinations thereof.

21. The method of claim 20, wherein the amount of the composition administered is effective to prevent or reduce loss of or reduction in memory, learning, attention, decision accuracy, decision speed, navigation skills, motor activity, or a combination thereof in the insect pollinator upon exposure to a pesticide.

22. The method of claim 20, wherein the amount administered is effective to prevent or reduce loss of or reduction in memory, learning, navigation skills, motor activity, or combinations thereof in the insect pollinator compared to an insect pollinator not administered the composition.

23. The method of claim 20, wherein the amount administered is effective to prevent or reduce mitochondrial dysfunction, apoptosis and/or oxidative stress in the brain.

24. The method of claim 20, wherein the amount administered is effective to prevent or reduce mitochondrial dysfunction, apoptosis and/or oxidative stress in the mushroom bodies.

25. The method of claim 20, wherein the impairment is induced by exposure to the pesticide.

26. The method of claim 25, wherein the pesticide is at a sublethal dose.

27. The method of claim 26, wherein the pesticide is neurotoxic to insect pollinators.

28. The method of claim 27, wherein the pesticide adversely affects GABAergic or glutamatergic neurotransmission or the function of mushroom bodies; increases mitochondrial dysfunction, apoptosis or oxidative stress in the brain; or combinations thereof.

29. The method of claim 27, wherein the pesticide targets the nicotinic acetylcholine receptor (nAChR).

30. The method of claim 27, wherein the pesticide is a neonicotinoid.

31. The method of claim 27, wherein the pesticide is imidacloprid, thiacloprid, clothianidin, thiamethoxam, acetamiprid, nitenpyram, dinotefuran, nithiazine, fipronil, carbamate, an organophophate, a derivative of sulfloxamine, or a pyrethroid, optionally wherein the pyrethroid is deltamethrin.

32. The method of claim 31, wherein exposure to the pesticide occurs after administration of the composition.

33. The method of claim 32, wherein the composition is a food, feed additive or supplement, or nutraceutical.

34. The method of claim 33, wherein the composition is administered via ingestion.

35. The method of claim 34, wherein the pesticide is delivered by foliar application, soil injection, tree injection, ground application as a granular or liquid formulation, or as a pesticide-coated seed treatment.

36. The method of claim 35, wherein the one or more phenolic compounds is quercetin, rutin, kaempferol, p-coumaric acid, or a combination thereof.

37. A composition suitable for administration to or ingestion by an insect pollinator comprising one or more phenolic compounds in an effective amount to protect against impairment of a cognitive function of the insect pollinator.

38. The composition of claim 37 further comprising a source of carbohydrates, proteins, lipids, vitamins, minerals, water or combinations thereof.

39. The composition of claim 38, wherein the source is natural or artificial nectar, honey, sugar, sugar syrup, pollen or pollen substitute, soy flour, soy meal, gluten, skim milk, yeast, pollard, oil, or combinations thereof.

40. The composition of claim 38, wherein the source is selected from the group comprising AP23®, BEE-PRO®, FEEDBEE, MEGABEE and ULTRA BEE.

41. The composition of claim 37, wherein the one or more phenolic compounds is one or more flavonoids, p-coumaric acid, or combination thereof.

42. The composition of claim 41, wherein the flavonoid is selected from the group comprising flavones, flavanones, flavonols, isoflavones, anthocyanins, flavanols (catechins), chalcones, and neoflavonoids.

43. The composition of claim 42, wherein the flavonol is selected from quercetin, rutin, myricetin, kaempferol, fisetin, morin, isorhamnetin, and derivatives or variants thereof.

44. The composition of claim 37, wherein the one or more phenolic compounds is selected from the compounds of Table 1 (e.g., isoquercetin, rutin, quercetin, quercitrin, hyperoside, quercimeritrin, baimaside, querciturone, 3-O-methylquercetin, quercetin 3-sambubioside, miquelianin, spiraeoside, isorhamnetin, quercetin 3-O-sulfate, quercetin 7-O-glucoside, quercetin 3-O-Rhamnoside, quercetin 3,4′-diglucoside, and quercetin 3-sophorotrioside) and combinations thereof.

45. The composition of claim 37, wherein the one or more phenolic compounds is selected from quercetin, rutin, myricetin, kaempferol, fisetin, morin, isorhamnetin, p-coumaric acid, and derivatives or variants thereof, and combinations thereof.

46. The composition of any one of claims 37-45 for use as a food, feed additive or supplement, or nutraceutical.

47. The composition of any one of claims 37-45, wherein the insect pollinator is a butterfly, moth, fly, beetle, wasp or bee.

48. The composition of claim 47, wherein the bee is selected from honeybee, bumblebee, carpenter bee, leafcutter bee, blueberry bee, squash bee, mason bee, orchid bee, stingless bee, or sweat bee.

49. The composition of claim 48, wherein the cognitive function is selected from learning, memory, attention, decision accuracy, decision speed, navigation, motor activity, sucrose sensitivity, and combinations thereof.

50. The composition of claim 49, wherein the impairment is induced by exposure to a pesticide.

51. The composition of claim 50, wherein the pesticide is neurotoxic.

52. The composition of claim 50, wherein the pesticide adversely affects GABAergic or glutamatergic neurotransmission, the mushroom bodies, the antennal lobes, the optic lobes or mitochondrial function; increases apoptosis; increases oxidative stress; or combinations thereof in the pollinator.

53. The composition of claim 50, wherein the pesticide targets the nicotinic acetylcholine receptor (nAChR).

54. The composition of claim 50, wherein the pesticide is a neonicotinoid.

55. The composition of claim 50, wherein the pesticide is imidacloprid, thiacloprid, clothianidin, thiamethoxam, acetamiprid, nitenpyram, dinotefuran, nithiazine, fipronil, carbamate, an organophophate, a derivative of sulfloxamine, or a pyrethroid, optionally wherein the pyrethroid is deltamethrin.

56. A method of protecting the cognitive function of an insect pollinator, the method comprising administering to an insect pollinator in need thereof a composition of any one of claims 37-45.

57. A method of protecting the learning and/or memory capabilities of an insect pollinator, comprising administering to the insect pollinator a composition of any one of claims 37-45.

58. A method for preventing or reducing impairment of cognitive function in an insect pollinator, comprising administering to the insect pollinator a composition comprising an effective amount of one or more phenolic compounds to prevent or reduce impairment of a cognitive function in the insect pollinator.
